(19) United States
(12) Patent Application Publication
(10) Pub. No.: US 2004/0171543 A1
(43) Pub. Date: Sep. 2, 2004

(54) Matrix metalloproteinase inhibitors and methods for identification of lead compounds

(76) Inventors: Anne-Claude Dublanchet, Fresnes Cedex (FR); Charles Andrianjara, Fresnes Cedex (FR); Roger Wrigglesworth, Fresnes Cedex (FR); Claude Bertrand, Fresnes Cedex (FR); Pierre Ducrot, Fresnes Cedex (FR); Renaud Morales, Fresnes Cedex (FR); Venkataraman Thanabal, Ann Arbor, MI (US); Alexander G. Pavlovsky, Ann Arbor, MI (US); Margaret M. O'Gara, Fresnes Cedex (FR); Jennifer M. Thorn, Ann Arbor, MI (US); Delphine Compere, Fresnes Cedex (FR)

Correspondence Address: PFIZER INC., PATENT DEPARTMENT, MS8260-1611, EASTERN POINT ROAD, GROTON, CT, 06340, US

(21) Appl. No.: 10/637,942

(22) Filed: Aug. 7, 2003

(30) Foreign Application Priority Data
Aug. 13, 2002 (WO) PCT/GB02/03728

(51) Int. Cl. A61K 38/17

(52) U.S. Cl. 514/12

(57) ABSTRACT

The invention relates to compounds that are selective inhibitors of matrix metalloproteinases, to pharmaceutical compositions containing them and to their use in the prevention and/or treatment of MMP-associated diseases. The invention also relates to methods for identification of lead compounds that are selective inhibitors of matrix metalloproteinases. The compounds have the properties that they:

(a) bind allosterically to a matrix metalloproteinase or small group of metallic metalloproteinases;

(b) bind into at least the S1′ pocket, at least the S1″ pocket (as defined) or at least the S1′ pocket and the S1″ pocket of said matrix metalloproteinase; and

(c) exhibit selectivity for a matrix metalloproteinase or group of matrix metalloproteinases other than MMP-13.

### FIELD OF THE INVENTION

The present invention relates to compounds that are selective inhibitors of matrix metalloproteinases, to pharmaceutical compositions containing them and to their use in the prevention and/or treatment of MMP-associated diseases. The invention also relates to methods for identification of lead compounds that are selective inhibitors of matrix metalloproteinases.

### BACKGROUND OF THE INVENTION

Matrix metalloproteinases (MMPs) are naturally-occurring enzymes found in most mammals and are zinc-dependent endopeptidases.

One major biological function of the MMPs is to catalyze the breakdown of connective tissue or extracellular matrix by virtue of their abilitity to hydrolyze various components of the tissue or matrix. Examples of the components that may be hydrolyzed by an MMP include collagens (e.g., type I, II, III, or IV), gelatins, proteoglycans, and fibronectins. Apart from their role in degrading connective tissue, MMPs are also involved in the activation of the zymogen (pro) forms of other MMPs thereby inducing MMP activation. They are also involved in the biosynthesis of TNF-alpha which is implicated in many pathological conditions and can cause/contribute to the effects of inflammation, rheumatoid arthritis, asthma, COPD, autoimmune disease, multiple sclerosis, graft rejection, fibrotic disease, cancer, infectious diseases, malaria, mycobacterial infection, meningitis, fever, psoriasis, cardiovascular/pulmonary effects (e.g., post-ischemic reperfusion injury), congestive heart failure, hemorrhage, coagulation, hyperoxic alveolar injury, radiation damage, cachexia, anorexia, and acute phase responses like those seen with infections and sepsis and during shock (e.g., septic shock and hemodynamic shock).

Over-expression and/or over-activation of a matrix metalloproteinase (“MMP”), or an imbalance between an MMP and a natural (i.e., endogenous) tissue inhibitor of a matrix metalloproteinase (“TIMP”), has been linked to the pathogenesis of diseases characterized by the breakdown of connective tissue or extracellular matrix. Examples of diseases characterized by over-expression and/or over-activation of an MMP include rheumatoid arthritis, asthma, COPD, osteoarthritis; osteoporosis; periodontitis; multiple sclerosis; gingivitis; corneal, epidermal, and gastric ulceration; atherosclerosis; neointimal proliferation, which leads to restenosis and ischemic heart failure; stroke; renal disease; macular degeneration; and tumor metastasis.

Further, some MMP-mediated diseases may involve overactivity of only one MMP enzyme. This is supported by the recent discovery that MMP-13 alone is over-expressed in breast carcinoma, while MMP-1 alone is over-expressed in papillary carcinoma.

Research has been carried out into the identification of inhibitors that are selective e.g. for a few of the MMP subtypes. A MMP inhibitor of improved selectivity would avoid potential side effects associated with inhibition of MMPs that are not involved in the pathogenesis of the disease being treated. Further, use of more selective MMP inhibitors would require administration of a lower amount of the inhibitor for treatment of disease than would otherwise be required and, after administration, partitioned in vivo among multiple MMPs. Still further, the administration of a lower amount of compound would improve the margin of safety between the dose of the inhibitor required for therapeutic activity and the dose of the inhibitor at which toxicity is observed.

The design and therapeutic application of matrix metalloproteinase inhibitors was reviewed by Whittaker et al., Chem Rev., 1999, 99, 2735-2776. The authors explained that the requirement for a molecule to be an effective inhibitor of the MMP class of enzymes is a functional group (e.g. carboxylic acid, hydroxamic acid or sulfhydryl) capable of chelating to the active site zinc II ion, at least one functional group that provides a hydrogen bond interaction with the enzyme backbone, and one or more side chains which undergo effective van der Waals interactions with the enzyme subsites. A large number of such compounds are mentioned in which chelation is by a hydroxamate group.

Chen et al., J. Am. Chem. Soc, 2000, 122, 9648-9654 disclose a potent and selective inhibitor for MMP-13. The authors had found that a compound referenced CL-82198 exhibited weak inhibition of MMP-13 but complete lack of activity against MMP-1, MMP-9 and TACE:

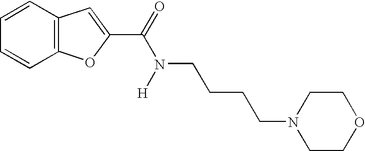

They postulated as a result of NMR and docking studies (see FIG. 4 of the above paper) that the above compound sits in and extends along the S1′ pocket of MMP-13, with the morpholine group forming a hydrogen bond with the backbone amide group of Leu-82 and with the benzofuran group packing deep into the S1′ pocket, but not binding to zinc of the catalytic domain. The authors decided that the way forward in the design of an MMP-13-selective lead compound was to make a compound that had both a moiety that chelates to zinc of the catalytic domain and a moiety that sits in the S1′ pocket, and arrived at a potent compound called WAY-170523 that shows >5800-, 56- and 500-fold selectivity against MMP-1, MMP-9 and TACE:

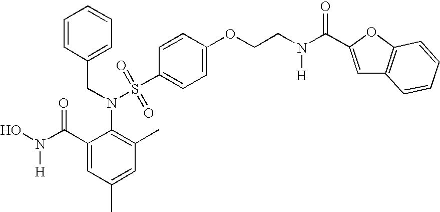

Other compounds that combine a first portion containing a functionality that forms a direct complex with the catalytic zinc atom in the active site of a matrix metalloproteinase and a second portion that is intended to sit in the S1′ pocket are described in WO 01/05389 (Stallings et. al., G. D. Searle). It is doubtful, however, whether this approach will lead to compounds of practical utility since complex formation is via an N-hydroxy group or a group closely related thereto located adjacent to an aryl ring, and such compounds have been reported to be carcinogenic or mutagenic, see Weisburger, J. H. et al., “Biochemical formation and pharmacological, toxicological and pathological properties of hydroxylamines and hydroxamic acids”, Pharmacol. Rev., 1973, 25(1), 1-66.

Further compounds that exhibit selectivity for MMP-12 are described in WO 01/83431 and WO 01/83461 (Shionogi) and are stated to be effective against emphysema and COPD. They rely for activity on the presence of groups that chelate to zinc.

Curtin et al., Bioorg. Med. Chem. Lett. 11 (2001), 1557-1560 disclose MMP inhibitors bearing a zinc-binding group, for example the compound below which was reported to be highly selective for MMP-2 versus MMP-1:

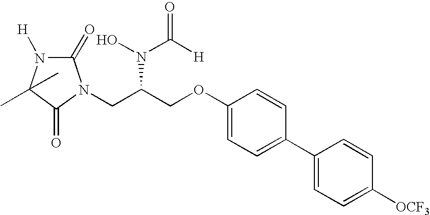

Wada et al, J. Med. Chem., 2002, 45, 219-232, working in the same laboratory, discovered a compound that is selective for the inhibition of MMP-2 and MMP-9 over MMP-1, and which demonstrated antitumor activity in a murine syngenetic tumor growth model:

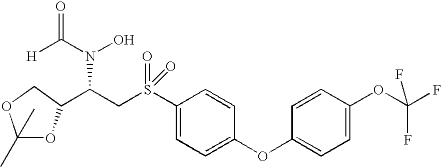

The authors attribute selectivity in MMPs to differences in the depth of the S1′ pocket and classify the MMPs into those with relatively deep pockets (MMP-2, -3, -8, -9, and -13) and those with shallow pockets (MMP-1 and -7). Selectivity is achieved by incorporation of an extended so-called P1′ group such as biphenyl for fitting into the S1′ pocket whereas the incorporation of smaller P1′ groups generally leads to broad-spectrum inhibition. Again, the above compounds achieve activity by the presence of groups that chelate to zinc.

There is a need for further inhibitors of MMPs that exhibit selectivity for individual enzymes or for groups of enzymes.

### SUMMARY OF THE INVENTION

We have identified a number of reasons for the poor selectivity and other disadvantages associated with existing MMP inhibitors:

MMP inhibitors typically mimic the natural substrates in that they coordinate the functional zinc cation and occupy from 1 to 3 specificity binding pockets along the enzyme active site cleft. As there is much structural similarity among these binding pockets of the various MMPs, this binding mode generally leads to the poor inhibitor-MMP selectivity.

Because they coordinate with the functional zinc cation of the MMP, existing inhibitors are competitive with binding of the endogenous substrate. As the concentration of an enzyme's substrate rises, the potency of a competitive inhibitor for the active site of the enzyme diminishes. This is a disadvantage for a pharmaceutical agent, as a rising concentration of substrate will eventually reduce the agent's therapeutic efficacy.

We consider that if MMP inhibitors bind allosterically to an enzyme or group of enzymes, then they should exhibit improved selectively because they do not employ the coordination to zinc that is a common feature amongst MMPs. Furthermore, a noncompetitive or uncompetitive MMP inhibitor could also bind to MMP-TIMP complexes and may not suffer diminishing binding potency in the presence of a rising concentration of substrate. Accordingly, a noncompetitive or uncompetitive MMP inhibitor that binds to an MMP-TIMP complex should maintain its therapeutic efficacy in the presence of a rising substrate concentration. A further advantage of a noncompetitive or uncompetitive MMP inhibitor is that when the inhibitor is bound to an MMP-TIMP complex and the TIMP disassociates from the complex to provide free TIMP and inhibitor-bound MMP, the MMP remains inhibited.

Our application No WO 02/06480 (PCT/IB 02/00447), the contents of which are incorporated herein by reference, provides compounds that bind allosterically into the S1 and S1″ binding sites of MMP-13, the S1″ binding site being newly discovered and being defined by residues from Tyr246 to Pro 255 as defined with reference to a sequence for MMP-13 that is included in the specification of that application. That pattern of binding offers the possibility of greater selectivity than is achieved with known inhibitors that bind to a catalytic zinc atom at the active site and the S1′ pocket. Note that in the alignment shown in FIG. 1 of this application this corresponds to Tyr 458 to Pro 467, and in the alignment of FIG. 2 of Terp et al., J. Med Chem., 45(13), 2675 (2002) it corresponds to Tyr 225 to Pro 235. The above numbers refer to the position of the amino acid in the sequence alignments of FIG. 1 of this application and of Terp, but do not match with the numbering in the sequence alone.

This invention is based on the realization that there exists a class of potent inhibitors selective for a single MMP other than MMP-13 or small group of MMPs, whose mode of binding is allosteric, and which bind into at least one and preferably both of the S1′ and S1″ binding sites. The existence of potent and selective allosterically binding inhibitors for MMP-13 and the similarity between the MMP enzymes supports the proposition that potent and selective allosterically binding inhibitors exist for other subtypes. We have already made inhibitors for MMP subtypes other than MMP-13 that are potent, but broad-spectrum. We have also made inhibitors that although selective for MMP-13 additionally exhibit selectivity for other subtypes (see Examples 1-3 below). Furthermore, our studies have revealed that MMPs other than MMP-13 can modify their conformation at the S1′ binding site in the presence of inhibitor to make available a S1″ binding site that corresponds to that observed for MMP-13, and compounds that bind to both the S1′ binding site and S1″ binding site are preferred on the ground of the increased selectivity available. In particular, we have found a number of compounds that bind allosterically to MMP-12.

In one aspect, the invention provides a compound that is a matrix metalloproteinase inhibitor, and that (a) binds allosterically to said matrix metalloproteinase, (b) binds into at least one and preferably both of the S1′ binding site and S1″ binding site of said matrix metalloproteinase, and (c) exhibits selectivity for a matrix metalloproteinase or group of matrix metalloproteinases other than MMP-13.

A compound meeting the above requirements may have a molecular weight in the range 350-550 and comprise 1-4 ring systems which are aryl except for one which may be partially or wholly saturated, the scaffold having typically 2-6 ring hetero atoms selected from N, S and O and optionally having carbonyl in one or two ring positions.

The compound may also have first and second linking groups having 1-4 chain atoms and may have at one end of the molecule a —COOH or >SO₂ group

The invention further provides a method of treating or preventing a disease associated with over-expression of one or more matrix metalloproteinases in a patient suffering from, or liable to suffer from, said disease, which comprises administering to said patient a compound as defined above.

The invention further provides a method of treating cancer associated with over-expression of MMP-2 and/or MMP-9 in a patient suffering from cancer, which comprises administering to said patient a compound as defined above.

The invention further provides a method of treating or preventing rheumatoid arthritis or osteoarthritis associated with over-expression of MMP-3 and/or MMP-9 in a patient suffering from, or liable to suffer from rheumatoid arthritis or osteoarthritis, which comprises administering to said patient a compound as defined above.

The invention further provides a method of treating or preventing chronic obstructive pulmonary disease and/or asthma associated with over-expression of MMP-9 and/or MMP-12 in a patient suffering from, or liable to suffer from chronic obstructive pulmonary disease and/or asthma, which comprises administering to said patient a compound as defined above.

The invention further provides a method of treating or preventing allergic rhinitis associated with over-expression of MMP-9 and/or MMP-12 in a patient suffering from, or liable to suffer from allergic rhinitis, which comprises administering to said patient a compound as defined above.

The invention further provides a method of identifying a compound as defined above or useful in a method as defined above, said identification method comprising:

docking the compound into the catalytic domain or domains of a target matrix metalloproteinase or group thereof; and

checking the availability of a binding mode in which said compound binds allosterically into an S1′ pocket, S1″ pocket or both.

The invention yet further provides a method of identifying a compound as defined above or useful in a method as defined above, said identification method comprising:

determining an IC₅₀ of said compound for a target matrix metalloproteinase inhibitor or group thereof other than MMP-13;

determining whether said compound exhibits selectivity for said target matrix metalloproteinase or group;

determining whether said compound binds allosterically.

The invention yet further provides a method of identifying a compound as defined above or useful in a method as defined above, said identification method comprising:

docking the compound into the catalytic domain or domains of a target matrix metalloproteinase or group thereof;

checking the availability of a binding mode in which said compound binds allosterically into an S1′ pocket, S1″ pocket or both;

determining an IC₅₀ said compound for a target matrix metalloproteinase inhibitor or group thereof other than MMP-13;

determining whether said compound exhibits selectivity for said target matrix metalloproteinase or group;

determining whether said compound binds allosterically, the sequence in which the docking and determining steps are performed being optional.

The above method may further include a crystallization step in which the compound and target matrix metalloproteinase are co-crystallized, followed by determination of the structure of the crystallized adduct by X-ray and/or NMR studies to reveal the mode of binding of the compound into the catalytic domain of the matrix metalloproteinase. Such further studies can provide information leading to the identification of a pharmacophore for the compound, and can confirm the feasibility of the method described above.

### BRIEF DESCRIPTION OF THE DRAWINGS

Various aspects of the invention and its relationship to the prior art will be described with reference to the accompanying drawings, in which:

FIG. 1 shows sequence alignments for MMPs 1-3, MMP-7, and MMPs 8-16 and identifies amino acids of the S1″ binding region;

FIG. 2 is a ribbon diagram of the catalytic domain of MMP-12 showing 4-({[4-(4-trifluoromethoxy-phenyl)-thiophene-2-carbonyl]-amino}-methyl)-benzoic acid docked allosterically into the S2″ and part of the S1′ binding sites;

FIG. 3 is a ribbon diagram of superimposed catalytic domains of MMP-12 and MMP-13 showing the zinc and calcium atoms associated with the catalytic domain, an inhibitor docked allosterically into the catalytic domain and differences in size and position of the S1′ loop between the two enzymes;

FIG. 4 is a ribbon diagram for the catalytic domain of MMP-13 (other MMPs are similar) showing a zinc atom of the binding site, the S1′ binding site to the left of the figure, the S1″ binding site to the right of the figure and a ligand docked to the enzyme and binding to the S1′ and S1″ binding sites, the numbering of the amino acids being that used in application No PCT/IB 02/00447;

FIG. 5 is a detail showing portions of helix A, helix B and loop 3 with the S1′ and S1″ binding sites also indicated;

FIG. 6 is a view of the catalytic domain of MMP-13 showing the S1, S1′ and S1″ binding sites or pockets, the arrangement of these sites being similar for other MMP types;

FIG. 7 is a view based on crystallographic coordinates of the catalytic domain of MMP-13 showing in superposition 4-({[4-(4-trifluoromethoxy-phenyl)-thiophene-2-carbonyl]-amino}-methyl)-benzoic acid [PD0331224] and 3-Benzyl-2,4-dioxo-1,2,3,4-tetrahydro-quinazoline-6-carboxylic acid benzyl ester [PD0307143] docked allosterically in the presence of AcNHOH which chelates to zinc of the S1 binding region;

FIG. 8 shows the results of an assay for allosteric binding between N-[(3-phenylisoxazol-5-ylmethyl)-aminothiocarbonyl]-benzamide and MMP-2, the reciprocal of initial reaction velocity V₀ being plotted against the reciprocal of concentration of substrate [S] at 6 inhibitor concentrations of from 0 nanomolar to 600 nanomolar; and

FIG. 9 shows a compound allosterically docked into the catalytic domain of human MMP-12 and MMP-12 and provides an indication of protein sequence similarity, the numbers differing from those og FIG. 1, but the rectangles A and B being located adjacent the rectangle at position 460 in FIG. 1, and FIGS. 10-12 views based on crystallographic coordinates of the catalytic domain of MMP-12 showing in superposition compounds docked allosterically in the presence of AcNHOH which chelates to zinc of the S1 binding region.

### DETAILED DESCRIPTION OF PREFERRED EMBODIMENTS

Matrix Metalloproteinases

The “matrix metalloproteinases” or “MMPs” to which this invention is applicable include all full length mammalian proteinases, or a truncated from thereof, or a catalytic domain thereof, that contain a functional metal cation in their active catalytic site. The invention is also applicable to all variants, analogs, orthologs, homologs, and derivatives of such proteinases provided they retain their ability to hydrolyze polypeptides and their functional metal cation in their catalytic active site. For recent reviews of MMPs, see Woessner and Nagase, (2000) “Matrix metalloproteinases and TIMPs”, Oxford University Press, Oxford; Doherty et al. (2002) Expert Opinion Therapeutic Patents 12(5): 665-707.

MMPs with a S1′ binding pocket are contemplated. Illustrating but not limiting examples of such MMPs with a S1′ binding pocket are MMP-3, MMP-9, MMP-12, MMP-13.

“MMP-associated disorder” which is treatable according to the invention encompasses all disorders in which the expression and/or activity of at least one MMP needs to be decreased irrespective of the cause of such disorders. Such disorders include, for example, those caused by inappropriate ECM degradation. Illustrative but not limiting examples of such MMP-associated disorders are:

cancer;

inflammatory disorders such as inflammatory bowel diseases, multiple sclerosis, glomerulonephritis, and uveorentinitis;

lung diseases such as chronic obstructive pulmonary disorder, asthma, acute lung injury, and acute respiratory distress syndrome;

dental diseases such as periodontal disease and gingivitis;

joint and bone diseases such as osteoarthritis and rheumatoid arthritis;

liver diseases such as liver fibrosis, cirrhosis and chronic liver disease;

fibrotic diseases such as pulmonary fibrosis, lupus, glomerulosclerosis, systemic sclerosis and cystic fibrosis;

vascular pathologies such as aortic aneurysm, atherosclerosis, hypertension, cardiomyopathy and myocardial infarction;

restenosis;

opthalmological disorders such as diabetic retinopathy, dry eye syndrome, macula degeneration and corneal ulceration;

wound healing disorders such as non healing ulcers, excessive scar formation;

tissue ulceration such as gastric ulcers and skin ulcers;

skin disorders such as psoriasis;

uterus and pregnancy-related disorders such as adenomyosis and pre-eclampsia;

disorders caused by pathogens such as HIV-1 infection, bacterial meningitis;

central nervous system disorders such as Alzheimer's disease;

neuroinflammatory disorders such as multiple sclerosis and acute neuroinflammation; and also

Marfan syndrome, invertebral disk degeneration, graft-versus-host disease and lupus.

Up to 28 MMPs have been characterized so far in humans and several major groups have been determined based on substrate specificity, and are believed applicable to the invention. Details are given below:

Collagenases: Usually Associated with Diseases Linked to Breakdown of Collagen-Based Tissue e.g. Rheumatoid Arthritis and Osteoarthritis

MMP-1 (also known as collagenase 1, or fibroblast collagenase), substrates collagen I, collagen II, collagen III, gelatin, proteoglycans. Over-expression of this enzyme is believed to be associated with emphysema, with hyperkeratosis and atherosclerosis, overexpressed alone in papillary carcinoma.

MMP-8 (also known as collagenase 2, or neutrophil collagenase), substrates collagen 1, collagen II, collagen III, collagen V, collagen VII, collagen IX, gelatin over-expression of which can lead to non-healing chronic ulcers.

MMP-13 (also known as collagenase 3), substrates collagen 1, collagen II, collagen III, collagen IV, collagen IX, collagen X, collagen XIV, fibronectin, gelatin, recently identified as being overexpressed alone in breast carcinoma. The applicants believe that an inhibitor for this enzyme would be effective in the treatment of breast cancer and arthritis.

MMP-18 (also known as collagenase 4).

Stromelysins.

MMP-3 (also known as stromelysin 1), substrates collagen III, collagen IV, collagen V, collagen IX, collagen X, laminin, nidogen, overexpression believed to be involved in atherosclerosis, aneurysm and restenosis.

MMP-10 (also known as stromelysin 2), substrates collagen III, collagen IV, collagen V, elastin, fibronectin, gelatin.

MMP-11 (also known as stromelysin 3), substrates serine protease inhibitors (Serpins).

MMP-12 (also known as metalloelastase, human macrophage elastase, or HME), substrates fibronectin, laminin, believed to play a role in tumour growth inhibition and regulation of inflammation and to play a pathological role in emphysema and in atherosclerosis, aneurysm and restenosis. The applicants believe that an inhibitor for this enzyme would be effective in the treatment of chronic obstructive pulmonary disorder (COPD) and/or asthma.

Matrilysins:

MMP-7 (also known as matrilysin), substrates collagen IV, elastin, fibronectin, gelatin, laminin.

MMP-26 (also known as matrilysin 2 or endometase), substrates denatured collagen, fibrinogen, fibronectin, vitronectin.

Gelatinases—inhibition believed to exert a favorable effect on cancer, in particular invasion and metastasis:

MMP-2 (also known as gelatinase A, 72 kDa gelatinase, basement membrane collagenase, or proteoglycanase), substrates collagen I, collagen II, collagen IV, collagen V, collagen VII, collagen X, collagen XI, collagen XIV, elastin, fibronectin, gelatin, nidogen, believed to be associated with tumor progression through specificity for type IV collagen (high expression observed in solid tumors and believed to be associated with their ability to grow, invade, develop new blood vessels and metastasize) and to be involved in acute lung inflammation and in respiratory distress syndrome.

MMP-9 (also known as gelatinase B, or 92 kDa gelatinase), substrates collagen I, collagen III, collagen IV, collagen V, collagen VII, collagen X, collagen XIV, elastin, fibronectin, gelatin, nidogen The above enzyme is believed to be associated with tumor progression through specificity for type IV collagen, to be released by eosinophils in response to exogenous factors such as air pollutants, allergens and viruses, to be involved in the inflammatory response in asthma and to be involved in acute lung inflammation and respiratory distress syndrome. The applicants believe that an inhibitor for this enzyme would be effective in the treatment of chronic obstructive pulmonary disorder (COPD) and/or asthma.

Membrane Type-MMPs.

MMP-14 (also known as membrane MMP or MT1-MMP) substrates MMP-2, collagen 1, collagen II, collagen III, fibronectin, gelatin, laminin.

MMP-15 (also known as MT2-MMP), substrates MMP-2, collagen 1, collagen II, collagen III, fibronectin, laminin nidogen.

MMP-16 (also known as MT3-MMP), substrates MMP-2, collagen 1, collagen III, fibronectin.

MMP-17 (also known as MT4-MMP), substrates fibrin(ogen), TNF-α.

MMP-24 (also known as MT5-MMP), substrate MMP-2, gelatin, fibronectin, chondroitin, dermitin sulfate proteoglycans.

MMP-25 (also known as MT6-MMP), substrate MMP-2, gelatin, collagen IV, fibronectin.

MMP-19 Subfamily:

MMP-19 (also known as Rasi-1), substrates MMP-9, gelatin, laminin-1, collagen IV, fibronectin.

MMP-28 also known as epilysin, substrate caesin.

Other MMPs:

MMP-20 (also known as enamelysin), substrate amelogenin.

MMP-23 (also known as femalysin), substrate gelatin.

3D-Structure of the MMPs and S₁′ and S1″ Binding Pockets:

The catalytic domains of MMPs are generally very similar with sequence similarities in the range of 50-88% and identity in the range of 33-79%. The common structural features include three α-helices and a β-sheet consisting of four parallel and one anti-parallel strand.

As previously explained, MMPs are zinc and calcium dependant, and all known structures contain two zinc ions and between one to three calcium ions. The active site is a cavity spanning the entire enzyme, and it has been shown that a substrate containing at least six amino acids is required for the proteolytic activity of MMPs; these six amino acid occupying the subsites S3-S3′ (notation according to Schechter and Berger—Biochem. Biophys. Res. Commun. 1996, 27, 157-162.). All MMP structures contain the common sequence motif HxGHxxGxxH where the three histidines coordinate the catalytic zinc ion.

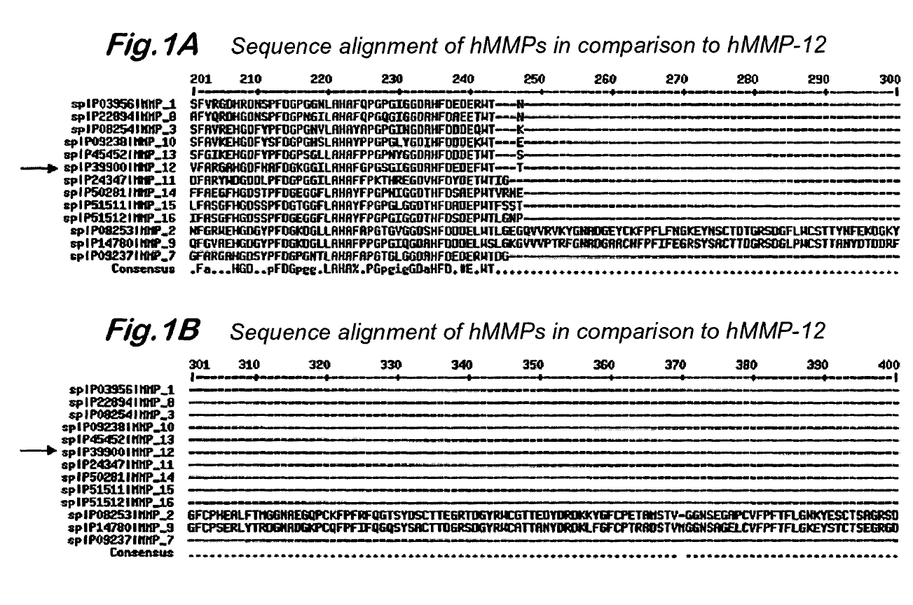

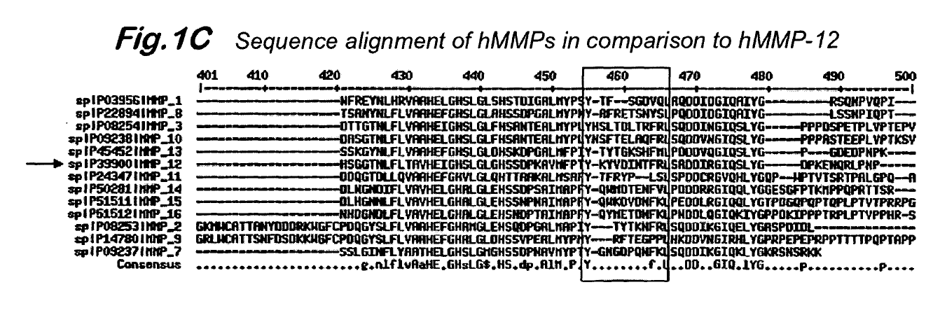

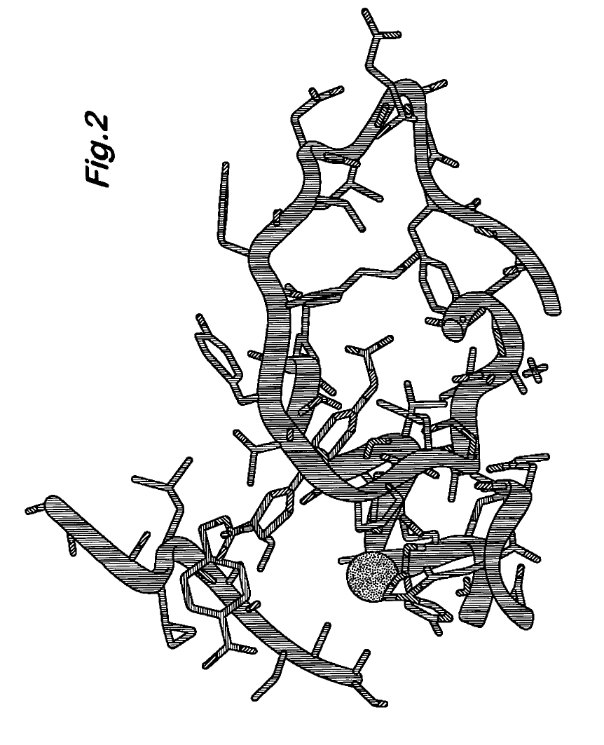

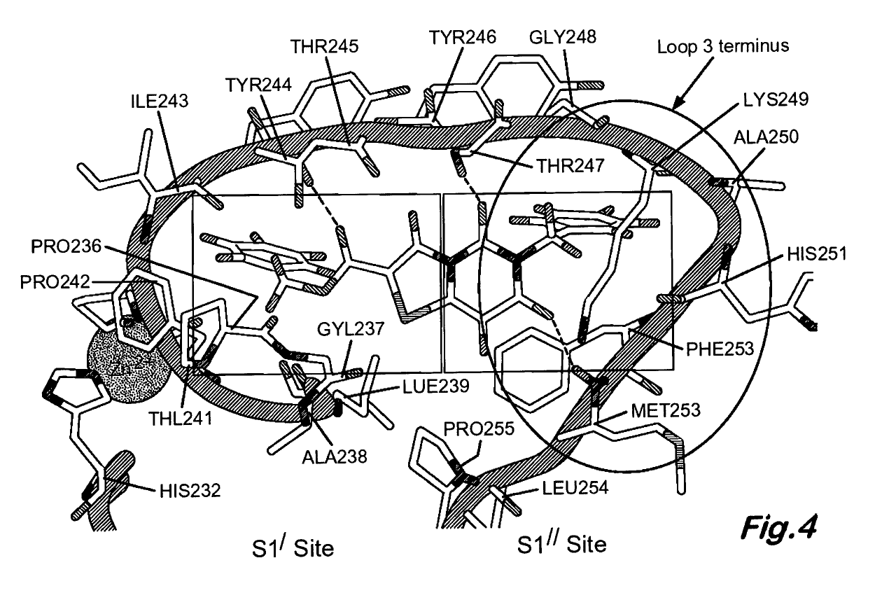

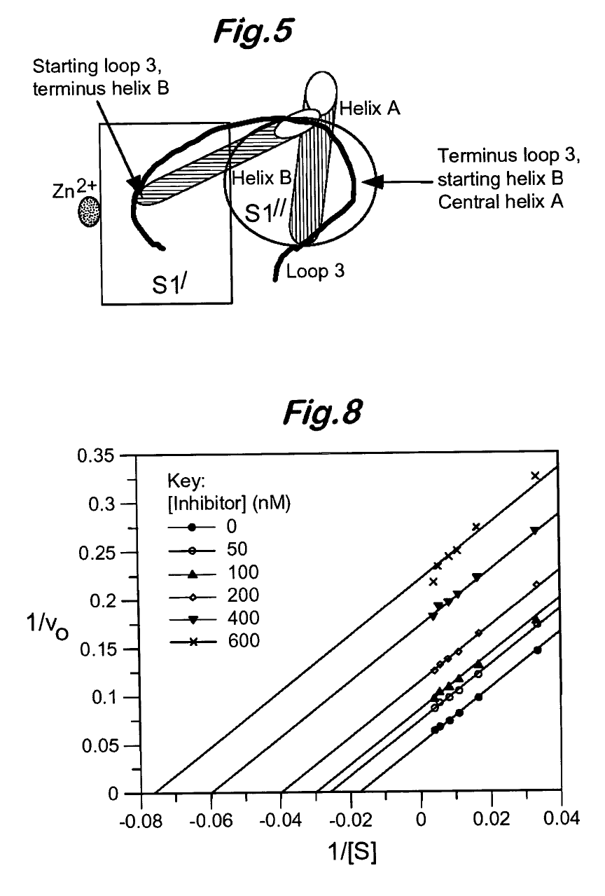

There is no clear definition of S1′ pocket of MMP in the literature. However, FIG. 1 is a sequence alignment for some 13 MMPs with the sequences appearing to the left of the red rectangle at residue 460 corresponding to the S1′ pocket (see also FIG. 2 of Terp et al., supra). The conventional definition below is derived from a collection of different articles, so it is more general than that will be found in any one of them. The pocket (see FIGS. 4 and 5) is surrounded by loop 3 which is of different length and amino acid composition in the various MMP enzymes. It extends through a hydrophobic channel constituted by the head of loop 3 and the end residues of helix B to a wall formed by the terminus of Loop 3 and the starting residues of helix B (despite the fact no compound has been known to interact with this loop3 terminus in the literature). The S1′ lateral side located in the core of the protein is made up of an hydrophobic wall formed by the association of helices A and B (FIG. 5). The other lateral side at the protein surface contains pores that are open to solvent. The size of the pores varies depending upon the MMP subtype. However, we prefer to regard the S1′ pocket as starting at the hydrophobic channel (constituted by the head of loop 3 and the terminus of helix B) and ending roughly at the middle part of loop 3. One lateral side is limited by the central segments of helices A and B located in the core of the MMPs. The other lateral side at the protein surface contains pores that are open to solvent. The size of the pore varies in MMP subtypes.

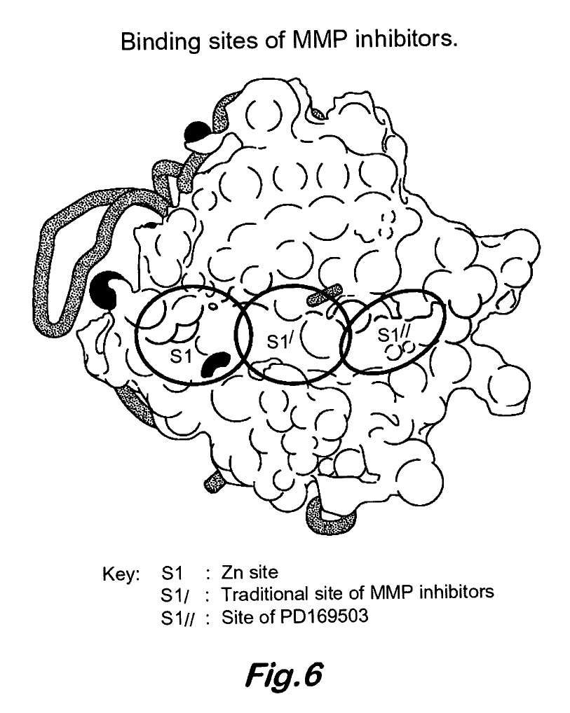

The S1″ pocket which has been newly reported by us (red rectangle in FIG. 1; see also FIGS. 4-6) is created by ligands that can bind into the enzyme by an induced-fit mechanism in a region constituted by the terminus of loop 3, the beginning of helix A and the terminus of helix B (FIG. 5). This region is charaterized by an hydrophobic channel formed by a homologous region (NFL) located at the start of helix B and surrounded by the terminus of loop 3. One end of the S1″ binding region is defined by the S1′ pocket (applicants' definition), and its other end is defined by a pore at the protein surface which is open to solvent and constituted by the start of Helix B, terminus of Loop 3 and a central part of helix A.

Most of the known MMP inhibitors are peptidomimetics and exert their function by coordinating to residues in the primed site. Regarding the strength of the inhibitors, the hydroxamates should intuitively be the best zinc chelating group, since this group has the best coordination geometry and proteolytic properties. This is in accordance with the experimental data, where carboxylic compounds are seen to be weaker inhibitors, and in addition, sulfur compounds are even weaker than these. In designing compounds for use in this invention, it is desirable to avoid the presence of zinc-chelating groups and in particular to avoid hydroxamate groups. Other groups e.g.—COOH have been included in compounds according to the invention without the binding mode involving chelation to zinc. The main type of selectivity that has been obtained up to now is for inhibition of the deep pocket enzymes over the short pocket enzymes, and this is achieved by the incorporation of an extended P1′ group (e.g. biphenyl) onto the Zn-chelating moiety, whereas generally speaking, the presence of smaller P1′ groups lead to broad-spectrum inhibition.

A co-crystal structure has been obtained between the catalytic domain of MMP-12 and the inhibitor CGS 27023A (J. Mol. Biol. 2001, 312, 743-751). In that structure CGS 27023A is bound to the catalytic zinc ion, as expected in a bidentate fashion, by the two hydroxamate oxygen atoms, resulting in a trigonal bipyramidal coordination of the zinc ion. The remainder of the ligand occupies the primed side of the active site cleft. The S1′ pocket is in fact a channel that connects the active-site cleft with the other side of the protein so that the observed ordered water molecules are in rapid exchange with bulk solvent through this channel. The bound conformation of the inhibitor is a low energy conformation in which the torsion angles about the rotatable bonds are close to stereochemically ideal values. The observed binding-mode of CGS27023A reveals that a favourable bound conformation, strong hydrogen bonding and hydrophobic interactions contribute to the strong affinity for MMP-12 (IC₅₀=2 nM). On the other hand, all interactions described can be formed equally well with various other members of the MMP-family, which in turn explains the missing or weak selectivity profile of CGS27023A.

In a first variant of the invention, selectivity for the target enzyme can be achieved by a combination of allosteric binding and binding into the S1′ pocket where readily recognizable differences between some of the MMP structures are found, see FIG. 1 and in particular the sequence differences to the left of or in the red rectangle that appears in that figure. The structure of the S1′ pocket in various MMPs with references for the numbering of the amino acids sequences is also disclosed in FIG. 2 of Terp et al, supra, at page 2677. It has not previously been proposed that selective and potent inhibitors of MMPs can be made that bind allosterically with the catalytic domain and into the S1′ pocket, achieve selectivity through differences in the S1′ pocket for the various MMPs.

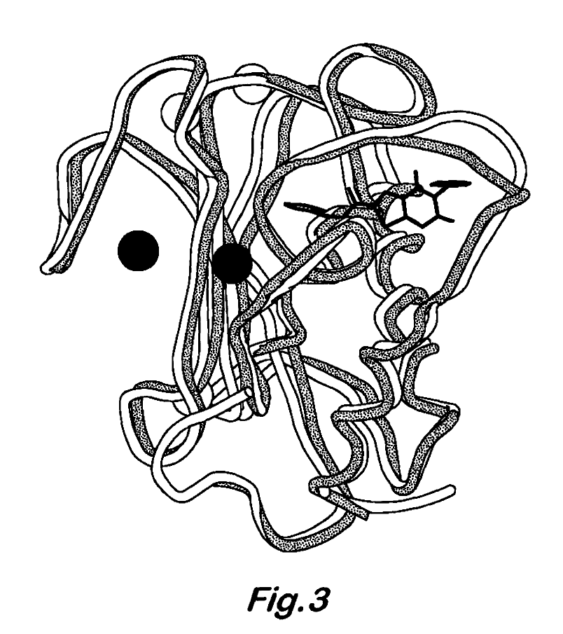

As previously explained, the S1′ pocket is surrounded by loop 3, which is of different length and amino acid composition in the individual MMPs. FIG. 3 is a ribbon diagram of the catalytic domains of MMP-12 and MMP 13, with the S1′ loop (loop 3) appearing at the upper right hand corner in the region marked B. The S1′ loop for MMP-13 is the upper loop and is larger and less rigid than the S1′ loop for MMP-12. Inhibitors that are effective for MMP-13 may therefore be unable to fit into the smaller S1′ site of MMP-12. There is therefore a possibility of exploiting such differences in the structures at the S1′ site of the various enzymes for selective purposes.

As recognized in the prior art from X-ray crystallographic analysis and homology modelling MMPs may be classified into two broad structural classes dependent on the depth of the S1′ pocket. This selectivity pocket is relatively deep for the majority of the enzymes (MMP-2, MMP-9, MMP-3, MMP-8, MMP-12, MMP-13, etc.) but for certain enzymes (MMP-1, MMP-7 and MMP-11) it is particularly or completely occluded.

Comparison of the sequence similarity between MMP-13 and MMP-1 illustrates the difficulty in designing specific MMP inhibitors. There are only a few significant residue differences between the two enzymes where these modifications result in a significant change in the local environment of the active-site. The R114 to L115 modification results in a conversion from a hydrophilic to a hydrophobic residue at the base of the S1′ pocket between MMP-1 and MMP-13 respectively. Then clearly, it is feasible to take advantage of these spatially distinct environmental changes between MMP-1 and MMP-13. Nevertheless, when these sequence and environmental differences are averaged across the MMP family it becomes less discriminating and extremely difficult to design an inhibitor to a specific MMP subtype based strictly on the small sequence differences.

Attention is drawn to the following features of particular enzymes that may be useful in designing selective compounds, the residue numbering being according to Terp et al, FIG. 2:

In MMP-1, an arginine defines the bottom of the pocket. MMP-1 could be targeted selectively because of its closed pocket (due to Arg197). Nevertheless it is interesting to note that MMP-1 can change its shape, making it accessible to more bulky substituents.

In MMP7, a tyrosine defines the bottom of the pocket, and permits an interaction with dry probe; MMP7 could be targeted selectively because of its closed pocket, and MMP-7 seems resistant to change in shape. This characteristic could be exploited to obtain selective inhibitors for MMP-7 versus MMP-1.

In all other MMPs for which NMR or X-ray structures are known (MMP-2, MMP-3, MMP-8, MMP-9, MMP-12, MMP-13, MMP-14 and MMP-20) the residue in this position (197), is either a leucine or a threonine (MMP-20) and the pocket adopts an extended shape.

MMP-12 presents a threonine at the position 198, which could authorize an interaction sp³ nitrogen atom with lone pair bonded to three heavy atoms. Another possible interaction exist with probes able to accept hydrogen bonds, like hydroxy group of phenol or carboxy (OH), and oxygen of sulfone/sulfoxide (OS); but this last possibility could be extends to all probes since the other proteins have hydrophobic residues at these positions. At the position 224, MMP-12 presents a lysine, which authorizes an interaction with OH or OS probes through positively charged residue.

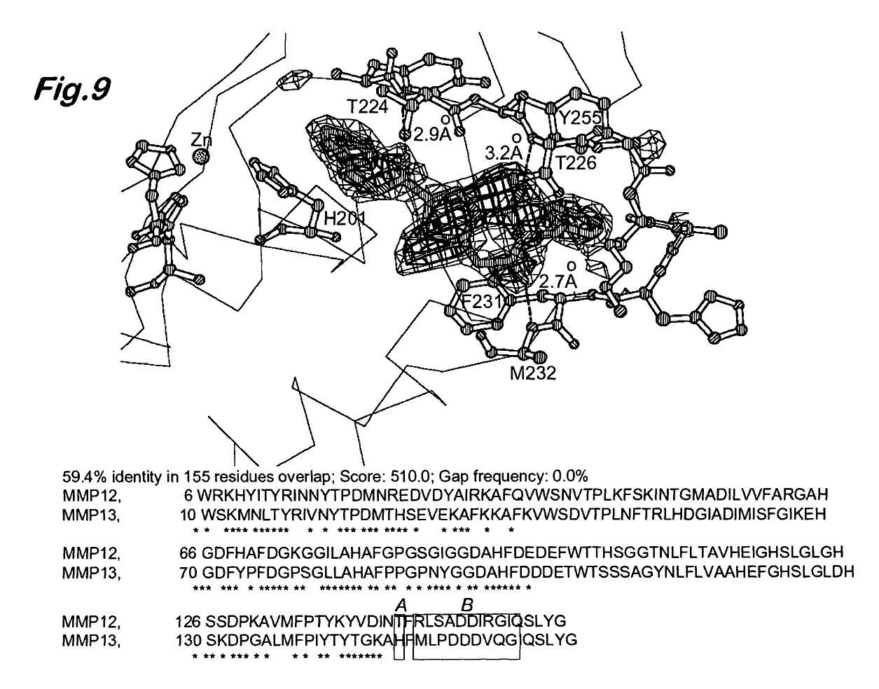

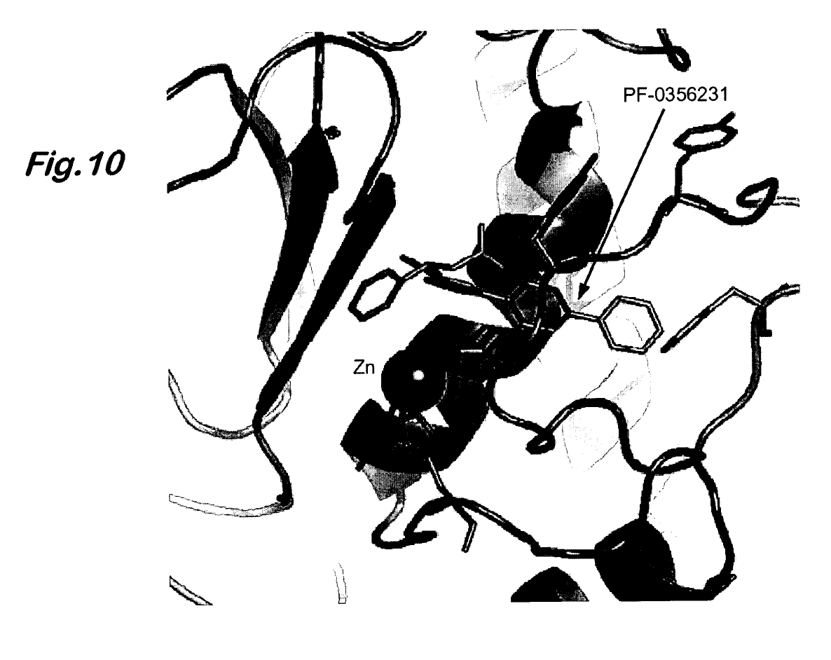

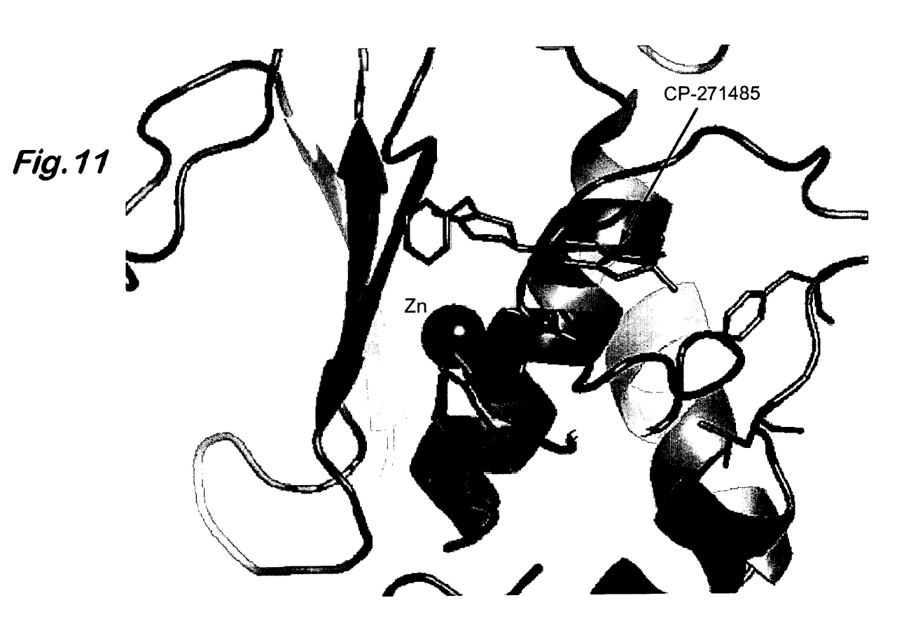

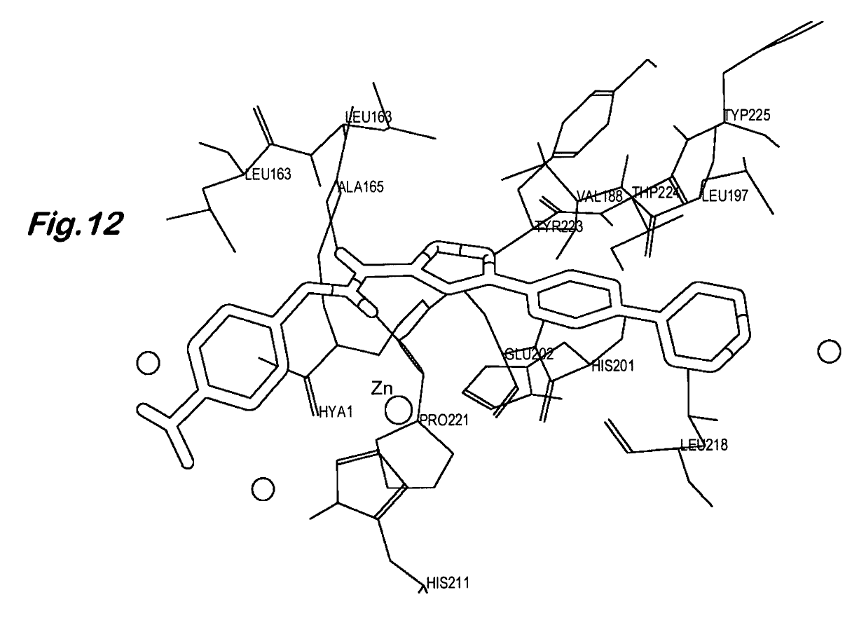

For comparison purposes, a feature of the MMP-13 structure is the large S1′ pocket relative to that of other MMPs, such as MMP-1, MMP-8 and matrilysin. The size of the S1′ pocket for MMP-13 is comparable with that of MMP-3 but differs in the overall shape of the pocket see also FIG. 9 which provides a comparison between portions of the sequences of human MMP-12 and MMP-13.

In a second variant of the invention, bonding of the inhibitor is in the S1′ and S1″ pockers. Flexibility of loop 3 opens up the possibility of accommodating ligands by an induced-fit mechanism, and this could not have been predicted from the rigid structure for these enzymes that was envisaged by Terp et al, supra. It has not previously been disclosed that MMPs can change conformation at the S1′ region on binding of an inhibitor to reveal the deeper S1″ binding region which is at positions corresponding to Tyr 246 to Pro 255 of MMP-13 as defined in PCT/IB 02/00447. In FIGS. 4-6 there is shown a view of the MMP 13 catalytic domain with the S1 site containing zinc, the S1′ site and the deeper S1″ sites marked: it is believed that the relative positions of these sites are similar for other MMPs. Compounds discovered by the applicants (e.g. as disclosed in the Examples of this application) can use this induced-fit mechanism to create the new S1″ pocket. The availability of the additional S1″ site provides opportunities to increase potency by binding into features of both pockets. It also provides opportunities to increase selectivity by taking account of differences in the two pockets.

Screening for Binding

Compounds of the invention desirably exhibit a potency demonstrated by an IC₅₀ for binding to its target matrix metalloproteinase or group of matrix metalloproteinases of less than 1 μM and preferably within the nM range. “IC₅₀” means the concentration of inhibitor required to inhibit the activity of an enzyme having a functional metal cation by 50% compared to the activity of the uninhibited enzyme or to the activity of the enzyme inhibited by a ligand to the functional metal cation.

The assays that can be used to evaluate the biological activity of various compounds for inhibiting MMPs are well-known and routinely used by those skilled in the art. They measure the amount by which a test compound reduces the hydrolysis of a thiopeptolide substrate caused by a matrix metalloproteinase enzyme. Such assays are described in detail by Ye et al., in Biochemistry, 1992;31(45): 11231-11235, which is incorporated herein by reference.

Thiopeptolide substrates show virtually no decomposition or hydrolysis in the absence of a matrix metalloproteinase enzyme. A typical thiopeptolide substrate commonly utilized for assays is Ac-Pro-Leu-Gly-thioester-Leu-Leu-Gly-OEt. A 100 μL assay mixture will contain 50 mM of 2-morpholinoethane sulfonic acid monohydrate (MES, pH 6.0) 10 mM CaCl₂, 100 μM thiopeptolide substrate, and 1 mM 5,5′-dithio-bis-(2-nitro-benzoic acid) (DTNB). The thiopeptolide substrate concentration is varied from 10 to 800 μM to obtain Km and Kcat values. The change in absorbance at 405 mm is monitored on a Thermo Max microplate reader (Molecular Devices, Menlo Park, Calif.) at room temperature (22° C.). The calculation of the amount of hydrolysis of the thiopeptolide substrate is based on E412=13600 m⁻¹ cm⁻¹ for the DTNB-derived product 3-carboxy-4-nitrothiophenoxide. Assays are carried out with and without matrix metalloproteinase inhibitor compounds, and the amount of hydrolysis is compared for a determination of inhibitory activity of the test compounds.

Selectivity

The compounds of the invention are required to be selective for a target MMP compared to other non targeted MMPs or to other control proteins that may be either structurally related to the MMP family (such as reprolysins or adamlysins such as ADAM-17 and ADAM-10) or not related to the MMP family. Since it may also be desirable to inhibit several MMPs in a given disorder, one skilled in the art may also be looking for compounds exhibiting selectivity for a small number of target MMPs. For example, compounds able to effectively inhibit both MMP-2 and MMP-9 are desirable in the prevention and/or treatment of cancer. Compounds able to effectively inhibit both MMP-3 and MMP-9 are desirable in the prevention and/or treatment of rheumatoid arthritis and osteoarthritis. Compounds able to effectively inhibit either MMP-12 or MMP-9 are desirable in the prevention and/or treatment of chronic obstructive pulmonary disease.

The selectivity of compounds for a single MMP target or for a small number of MMP targets is preferably 10 to 50-fold, more preferably 51 to 100-fold, even more preferably 101 to 1000-fold, even more preferably at least 1001-fold compared to other non targeted MMPs.

Selectivity may be determined using any method known to those skilled in the art. In such methods generally, the effect of different concentrations of a given compound on the activity of non-targeted MMPs and/or other control proteins are determined. The IC₅₀ values for the different MMPs/other proteins are then compared to the IC₅₀ value obtained (without any ligand for the functional metal cation) for the same compound for the targeted MMP(s). As an illustration, Binding Example 7 in Appendix A describes selectivity assays that may be used to assess selectivity towards different MMPs.

Allosteric Binding

As previously explained allosteric binding involves binding into the catalytic domain of MMPs but not forming a chelate bond that is competitive with endogenous ligand binding. This binding mode is illustrated in FIG. 5 where the compound 4-({[4-(4-trifluoromethoxy-phenyl)-thiophene-2-carbonyl]-amino}-methyl)-benzoic acid PD0307143] is shown docked into the S2′ and part of the S1′ binding site of MMP-13 in the presence of AcNHOH. which chelates to zinc of the S1 binding region.

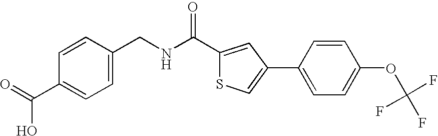

Also shown is compound PD0307143 which fits into the S1′ and S1″ (lowermost right as viewed in FIG. 5).

Allosteric binding may be demonstrated by the comparing the IC₅₀ of putative allosteric MMP inhibitors in the presence or absence of a ligand that chelates readily to zinc but is a sufficiently small molecule that it does not substantially interfere with the binding of the inhibitor to its intended location adjacent to the catalytic domain. Although ligand size is not critical, a bulky ligand will block more binding sites of the MMP than a smaller ligand, and may thus prevent potential inhibitors that bind close to, but not at, the functional metal cation from being identified. Suitable ligands are therefore low molecular weight molecules, e.g. acetohydroxamic acid (CH₃CONHOH) whose use is preferred.

How allosteric binding can be assayed is discussed in Appendix 1 which also gives examples of binding assays. It may be noted that compounds that bind allosterically can exhibit good potency, see e.g. Binding Example 2, compound 9 in Table 1 and Binding Example 6, and also that in many instances the acetohydroxamic acid and the inhibitor bind synergistically.

Identification of Lead Compounds by Molecular Modeling

As indicated above there is considerable information about the binding pockets in a number of the MMP enzymes. However, additional information may be obtained using the following programs:

SYBYL Matchmaker, available from Tripos of St Louis, Mo., USA which provides for prediction of 3D protein structure from sequence information.

SYBYL Composer, also available from Tripos, which enables protein models to be constructed automatically using knowledge-based homology modeling methods.

Homology, Modeler and Biopolymer from Accelrys which enable 3D protein sequences to be predicted and the resulting structures to be refined.

Lead compounds can be generated on the basis of the known structures of the S1′ and/or S1″ binding pockets using either structure-based drug design or ligand-based design, see J. Med. Chem., 1999, 42(22), 450.

The starting point for structure-based drug design is crystallographic data for a co-crystal of an enzyme and inhibitor, which provides information about affinity and selection types, and identifying and locating e.g electrostatic interactions, hydrophobic groups and hydrogen bond donors and acceptors useful in constructing a pharmacophore. Programs which are useful in such design include:

FlexX, available from Tripos, which for a protein with a known three-dimensional structure and a small ligand molecule can provide a prediction of the geometry of the protein-ligand complex and an estimate of the binding affinity and is therefore useful in virtual screening of compounds.

CScore, available from Tripos which uses multiple approaches for the evaluation of ligand-receptor interactions and combines individual scoring functions to produce a consensus score that is a more accurate indication of binding affinity.

Gold, available from CDCC, Cambridge, UK, which provides a prediction of how a flexible molecule will bind to a protein.

Ligand-based drug design can employ 2D and 3DQSAR and COMFA (Tripos), together with docking programs e g FlexX.

Bioavailability Assays

To be effective as a treatment, the compounds of the invention should preferably be orally bioavailable, i.e. a relatively high proportion of an orally administered drug should reach the systemic circulation. The factors that determine oral bioavailability of a drug are dissolution, membrane permeability and metabolic stability. Typically, a screening cascade of firstly in vitro and then in vivo techniques is used to determine oral bioavailability.

Dissolution, the solubilization of the drug by the aqueous contents of the gastro-intestinal tract (GIT), can be predicted from in vitro solubility experiments conducted at appropriate pH to mimic the GIT. Preferably the compounds of the invention have a minimum solubility of 10 IC₅₀. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro log D_{7.4} measurements using organic solvents and buffer. The log D can be determined by standard procedures known in the art such as described in J. Pharm. Pharmacol. 1990, 42:144. Cell monolayer assays such as CACO2 add substantially to prediction of favourable membrane permeability in the presence of efflux transporters such as p-glycoprotein, so-called caco-2 flux. The caco2 flux value can be determined by standard procedures known in the art such as described in J. Pharm. Sci, 1990, 79, 595-600.

Metabolic stability addresses the ability of the GIT or the liver to metabolize compounds during the absorption process: the first pass effect. Assay systems such as microsomes, hepatocytes etc are predictive of metabolic liability. Preferably the compounds of the Examples show metabolic stability in the assay system that is commensurate with an hepatic extraction of less then 0.5. Examples of assay systems and data manipulation are described in Curr. Opin. Drug Disc. Devel., 201, 4, 3644, Drug Met. Disp., 2000, 28, 1518-1523

Because of the interplay of the above processes, further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp., 2001, 29, 82-87; J. Med Chem, 1997, 40, 827-829, Drug Met. Disp., 1999, 27, 221-226.

In Vivo Effectiveness

Selected selective allosteric inhibitors able to bind to the S′1 and/or S1″ pocket of MMPs may be tested in appropriate pharmacological models such as those described in Appendix B, Reference Examples 8 to 9 in order to determine their potential effect on actual disease conditions and to select preferred compounds for development activities such as clinical trials. For example, animal models such as smoking mice and LPS-stimulated mice may be used to evaluate the inhibitory activity of such selective allosteric MMP inhibitors in COPD. Furthermore, potential side effects such as musculoskeletal side effects and local tolerability may also be evaluated using models such as those described in Reference Examples 8 and 9.

Dosage and Uses

It will be appreciated that the amount of compound of the invention and, optionally, further active constituents required for the treatment and/or prevention of a MMP-associated disorder will vary according to the route of administration, the disorder to be treated, the condition, age, the file history of the subject, and the galenic formulation of the pharmaceutical composition, etc. When treating a patient diagnosed as having a pathological condition influenced by the action of MMPs, the amount of compound of the invention is preferably effective when it provides at least partial inhibition of the target MMP or MMPs.

Depending upon its individual activity, a therapeutically effective dose of a compound of the invention will normally be from 1-500 mg/kg of body weight per day. Typical adult doses will normally be about 50 to about 500 mg per day. The quantity of active component in a unit dose preparation may be varied or adjusted from about 0.1 mg to about 500 mg, preferably about 0.5 mg to about 100 mg according to the particular application and the potency of the active compound. The composition can if desired contain other compatible therapeutic agents. A subject in need of treatment with a compound of the invention may be administered a dosage of about 0.1 to about 500 mg per day, either singly or in multiple doses over a 24 hour period.

If the pharmaceutical composition comprises further active constituents, in addition to the compound according to the invention, such further active constituents may be in the same composition for administering in combination concurrently, or in different compositions for administering substantially simultaneously but separately, or sequentially. In case of sequential administration, the further active ingredients may be administered prior or subsequently to the administering of the compound of the invention.

### EXAMPLE 1

4-{6-[3-(4-Methoxy-phenyl)-prop-1-ynyl]-4-oxo-4H-quinazolin-3-ylmethyl}-benzoic acid

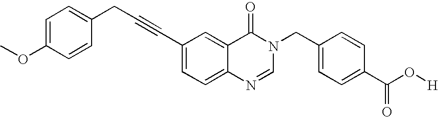

Step 1: 6-iodo-1H-benzo[a][1,3]oxazine-2,4-dione

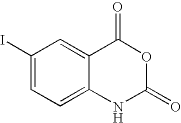

Dioxane (50 ml) was added to a suspension of 2-amino-5-iodobenzoic acid (4.9 g, 18.0 mmol) in H₂O (20 ml) and concentrated HCl (5 ml) was added until a clear solution was obtained. Neat diphosgene (5.95 g, 30.0 mmol) was added dropwise (with cooling at times so that the solution would not boil) to give a white precipitate. After stirring at room temperature for 10 min., water (ca. 100 ml) was added and the precipitate was filtered and washed with copious amount of water. It was then dried in vacuo to provide the desired product (5.2 g, quantitative) as white crystals.

N.M.R (DMSO-d₆) ¹H δ (ppm): 6.9 ( [symbol] [symbol] =8.6 Hz, 1H), 8.00 (dd, J=8.6, 2.0 + 1H), 8.10 ( [symbol] [symbol] =2.0 Hz, 1H), 11.8 (s, 1H); MS (APCI), M/z 288.0 (M−1)

Step 2: 2-Amino-5-iodo-benzamide

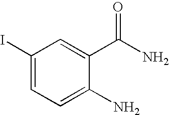

2.0 g (6.90 mmol) of the compound obtained in the preceding step was dissolved in approximately 50 ml of DMF, and an excess of aqueous ammonium hydroxide was added. After 10 minutes of stirring, the reaction solution was poured into 100 ml of water, and acidified with concentrated HCl, then extracted with 2×100 ml of EtOAc. The combined organic layer was then concentrated to yield 1.8 g (100%) of the desired product as an off-white powder.

N.M.R (DMSO-d₆) ¹H δ (ppm): 6.50 (d, J=8.8 Hz, 1H), 6.68 (s, 2H), 7.12 (s, 1H), 7.33 (dd, J₁=8.8 Hz, J₂=2.1 Hz, 1H), 7.77 (d, J=1.9 Hz, 2H).

Step 3: 6-Iodo-3H-quinazolin-4-one

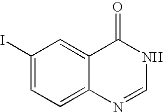

1.8 g (6.90 mmol) of the compound obtained in the preceding step was suspended in 30 ml of triethyl orthoformate. A catalytic amount of para-toluene sulfonic acid was added, and the suspension was refluxed for 3 hours. All volatiles were removed in vacuo, and the residue was washed with 1:1 dichloromethane:hexane to yield 1.5 g (80%) of an off white powder as the desired product.

N.M.R (DMSO-d₆) ¹H δ (ppm): 7.42 (d, J=8.5 Hz, 1H), 8.09 (dd, J₁=8.5 Hz, J₂=2.2 Hz, 1H), 8.09 (s, 1H), 8.34 (d, J=2.2 Hz, 1H), 12.38 (broad s, 1H); MS(APCI), M/z 270.9 (M−1);

Step 4: tert-Butyl 4-(6-Iodo-4-oxo-4H-quinazolin-3-ylmethyl)-benzoate

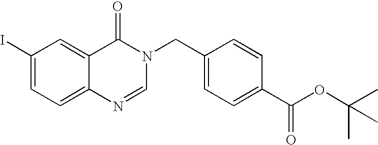

0.9 g (3.31 mmol) of the compound obtained in the preceding step was dissolved in 50 ml of DMF. 1.18 g (3.64 mmol) of cesium carbonate and 0.986 g (3.64 mmol) of t-butyl 4-bromomethyl-benzoate were then added. The reaction mixture was stirred at room temperature for 24 hours. 200 ml of EtOAc were then added, and the organic layer was washed with 3×100 ml of water, dried over MgSO₄ and concentrated. The residue was purified on a silica gel column using 4:1 dichloromethane:hexane increasing gradually to a 1:1 ratio, to yield 0.97 g (62%) of white powder as the desired product.

N.M.R (CDCl₃) ¹H δ (ppm) 5.21 (s, 2H), 7.36 (d, J=8.5 Hz, 2H), 7.43 (d, J=8.5 Hz, 1H), 7.96 (dd, J₁=6.6 Hz, J₂=3.1 Hz, 2H), 8.01 (dd, J₁=6.5 Hz, J₂=2.1 Hz, 1H), 8.07 (s, 1H), 8.64 (d, J=1.8 Hz, 1H); MS(APCI), M/z 270.9 (M−1).

Step 5: tert-Butyl 4-{4-oxo-6-[3-(4-methoxyphenyl)-prop-1-ynyl]-4H-quinazoline-3-ylmethyl}-benzoate

3.0 g (6.48 mmol) of the compound obtained in the preceding step was dissolved in 50 ml of DMF. 3.34 g (25.9 mmol) of diisopropylethylamine, catalytic amount of copper(I) iodide, 25.9 mmol 1-methoxy-4-prop-2-ynyl-benzene and catalytic amount of Pd(PPh₃)₂Cl₂ were then added in that order. The reaction solution was heated to 50° C. for 24 hours, then diluted with 300 ml of EtOAc and washed with 3×200 ml of water, 1×200 ml of brine. The organic layer was dried over MgSO₄ and concentrated. The residue was purified on a silica gel column with 4:1 hexane:EtOAc gradually increasing to 1:1 hexane:EtOAC to yield a waxy substance as the desired product.

N.M.R (DMSO-d₆) ¹H δ (ppm): 1.50 (s, 9H), 5.24 (s, 2H), 7.42 (d, J=8.8 Hz, 2H), 7.49 (d, J=8.6 Hz, 1H), 7.84 (d, J=8.6 Hz, 2H), 8.11 (dd, J₁=8.6 Hz, J₂=2.2 Hz, 1H), 8.39 (d, J=2.0 Hz, 1H), 8.59 (s, 1H).

Step 6: 4-[6-[3-(4-Methoxy-phenyl)-prop-1-ynyl]-4-oxo-4H-quinazoline-3-ylmethyl]-benzoic acid (Title Compound)

An excess (20 ml) of trifluroacetic acid was added to the compound obtained in the preceding step. After stirring for 30 minutes, all volatiles were removed and the residue triturated with 1:1 hexane:EtOAc. The precipitate was collected via filtration and washed with a small amount of methanol to yield the desired product as an off-white solid.

N.M.R (DMSO-d₆) ¹H δ (ppm): 3.70 (s, 3H), 3.83 (s, 2H), 5.24 (s, 2H), 6.89 (d, J=8.5 Hz, 2H), 7.29 (d, J=8.3 Hz, 2H), 7.41 (d, J=8.0 Hz, 2H), 7.65 (d, J=8.3 Hz, 1H), 7.81 (dd, J₁=8.3 Hz, J₂=1.5 Hz, 1H), 7.88 (d, J=8.1 Hz, 2H), 8.08 (d, J=1.5 Hz, 1H), 8.58 (s, 1H), 12.94 (broad s, 1H).

IC₅₀ values (μM) for the above compound were measured by TPL assays against the matrix metalloproteases indicated below, with the results indicated:

| | |
|---|---|
| MMP-1 | >100 |
| MMP-2 | >100 |
| MMP-3 | 0.22 |
| MMP-9 | >30 |
| MMP-12 | >30 |
| MMP-13 | 0.00014 |
| MMP-14 | >100 |

### EXAMPLE 2

4-{6-[3-(4-Methoxy-phenyl)-prop-1-ynyl]-1-methyl-2,4-dioxo-1,4-dihydro-2H-quinazolin-3-ylmethyl}-benzoic acid

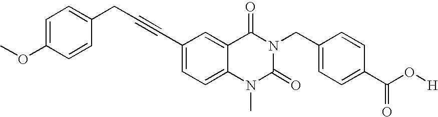

Step 1: Methyl 4-[(2-amino-5-iodo-benzoylamino)-methyl]-benzoate

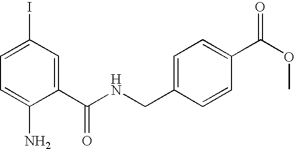

To a stirred solution of 15 g (74.4 mmol) of methyl 4-(aminomethyl)benzoate hydrochloride, 300 ml of dimethylformamide and 10.3 ml (7.53 g, 74.4 mmol) of triethylamine were added, at room temperature, followed by 10.06 g (74.4 mmol) of 1-hydroxybenzotriazole hydrate, 19.6 g (74.4 mmol) of 2-amino-5-iodobenzoic acid and 14.3 g (74.4 mmol) of 1-(3-dimethylaminopropyl)-3-ethyl carbodiimide hydrochloride. After stirring at room temperature overnight, the mixture was concentrated and the residue was dissolved in 300 ml of dichloromethane. The organic phase was washed with 150 ml water, 150 ml HCl 1N, and 150 ml water, dried over sodium sulfate and concentrated. The residue was recrystallized from 170 ml acetonitrile to afford after filtration 19.6 g (70%) of the desired product.

N.M.R: DMSO ¹H δ (ppm): 3.8 (s,3H), 4.45 (d,2H), 6.5-6.6 (m,3H), 7.3-7.45 (m,3H), 7.8-7.95 (m,3H), 8.9 (t,1H); Purity (HPLC): 99.1%

Step 2: Methyl 4-(6-iodo-2,4-dioxo-1,4-dihydro-2H-quinazolin-3-ylmethyl)-Benzoate

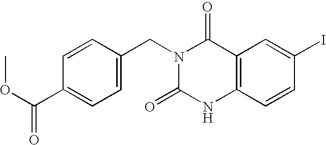

To a solution of 21.35 g (52 mmol) of the compound obtained in step 1 in 400 ml of dry tetrahydrofuran were added 9.3 g (57.2 mmol) of 1,1′-carbonyldiimidazole. The solution was heated overnight to 60° C. After cooling the precipitate was filtered and dried to afford 19.6 g (68.3%) of the desired product.

N.M.R: DMSO ¹H δ (ppm): 3.8 (s,3H), 5.1 (s,2H), 6.95-7.05 (m,1H), 7.35-7.45 (m,2H), 7.8-7.90 (m,2H), 7.9-8.0 (m,1H), 8.2 (s,1H), 11.6 (bs,1H); Purity (HPLC): 99.5%

Step 3: Methyl 4-(6-iodo-1-methyl-2,4-dioxo-1,4-dihydro-2H-quinazolin-3-ylmethyl)-benzoate

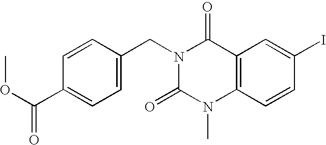

To a stirred suspension of 11 g (25.2 mmol) of the compound obtained in step 2 and 110 ml of dry DMF were added 5.22 g (37.8 mmol) of K₂CO₃, at room temperature. After 15 minutes, 7.85 ml (17.9 g, 126 mmol) of iodomethane were added. The reaction mixture was stirred for 2 hours and the precipitate filtered off and dissolved in a mixture of dichloromethane/methanol. The organic phase was washed with water, dried over Na₂SO₄ and concentrated to afford a precipitate corresponding to the desired product (10.1 g; 89%).

N.M.R: DMSO 1H δ (ppm): 3.5 (s,3H), 3.8 (s,3H), 5.2 (s,2H), 7.30 (d,1H), 7.45 (d,2H), 7.90 (d,2H), 8.1 (d,1H), 8.3 (s,1H); Purity (HPLC): 96.7%

Step 4: 4-(6-Iodo-1-methyl-2,4-dioxo-1,4-dihydro-2H-quinazolin-3-ylmethyl)-benzoic acid

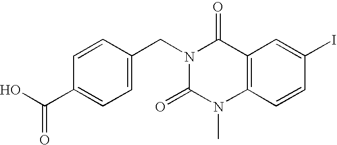

A mixture of 3.0 g (6.66 mmol) of the compound obtained in step 3, 30 ml of dioxane, 120 ml H₂O, and 0.56 g (13.3 mmol) of LiOH.H₂O was heated to reflux over 1 hour. After cooling and acidification with concentrated hydrochloric acid, the precipitate obtained was filtered off and recrystallized in dioxane/ether to afford 1.85 g (64.2%) of the desired product.

N.M.R: DMSO ¹H δ (ppm): 3.5 (s,3H), 5.2 (s,2H), 7.30 (d,1H),7.40 (d,2H), 7.85 (d,2H), 8.1 (d,1H), 8.30 (s,1H), 12.9 (bs, 1H); Purity (HPLC): 98.0%

Step 5: 4-{6-[3-(4-Methoxy-phenyl)-prop-1-ynyl]-1-methyl-2,4-dioxo-1,4-dihydro-2H-quinazolin-3-ylmethyl}-benzoic acid (Title Compound)

To a stirred solution of 0.68 g (1.56 mmol) of compound obtained in step 4 in 6.8 ml of dry DMF, were added successively, under nitrogen atmosphere, 1.2 ml (0.8 g, 6.24 mmol) of diisopropylethylamine, 56.8 mg (0.078 mmol) of dichlorobis (triphenylphosphine)palladium (II), a catalytic amount of CuI and 2.18 mmol of 3-(4-methoxyphenyl)-prop-1-ynyl The reaction mixture was heated to 50° C. over approximately 4 hours. Then, the mixture was concentrated under vacuum and the residue purified by flash chromatography (90:10 dichloromethane:methanol) to afford, after crystallization in dioxane the desired product.

TLC: CH₂Cl₂:MeOH 9:1 v/v Rf=0.50; N.M.R: DMSO ¹H δ (ppm): 3.55 (s,3H), 3.75 (s,3H), 3.8 (s,2H), 5.15 (s,2H), 6.9 (d,2H), 7.30 (d,2H), 7.40 (m,3H), 7.85 (m,3H), 8.00 (s,1H), 12.85 (bs,1H); IR: 2646, 1687, 1659, 1508, 1477, 1422, 1325, 1242, 1177, 1040, 950, 812 cm⁻¹ Mp=262° C.; Purity (HPLC): 95.4%

IC₅₀ values (μM) for the above compound were measured against the matrix metalloproteases indicated below using TPL assays, with the results indicated:

| | |
|---|---|
| MMP-1 | >100 |
| MMP-2 | >100 |
| MMP-3 | 0.83 |
| MMP-9 | >30 |
| MMP-12 | >100 |
| MMP-13 | 0.00018 |
| MMP-14 | >100 |

### EXAMPLE 3

2,4-Dioxo-3-[4-(2H-tetrazol-5-yl)-benzyl]-1,2,3,4-tetrahydro-thieno[2,3-d]pyrimidine-6-carboxylic acid 3-methoxy-benzyl ester

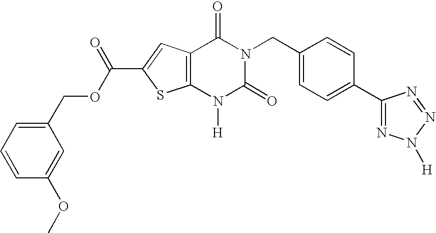

The above compound is prepared as described in WO 02/064598 (PCT/IB02/00204), the content thereof is herein incorporated by reference.

To a solution of 3-(4-cyano-benyzl)-2,4-dioxo-1,2,3,4-tetrahydro[2,3-d]pyrimidine-6-carboxylic acid benzyl ester (0.615 g, 1.42 mmol) in 10 mL of dioxane was added tributyltin azide (0.71 g, 2.14 mmol). The reaction solution was refluxed overnight. After removing the solvent in vacuo, the residue was dissolved in ether and HCl gas was bubbled in for 1 hour. The precipitate was filtered, dissolved in chloroform and chromatographied using EtOAc and THF. The fractions were collected and concentrated. The residue was triturated with 4:1 Hexane:EtOAc, to yield the 2,4-dioxo-3-[4-(2H-tetrazol-5-yl)-benzyl]-1,2,3,4-tetrahydro-thieno[2,3-d]pyrimidine-6-carboxylic acid 3-methoxy-benzyl ester as an off white solid (7%).

MS (APCI+), m/z 491 (M+1).

IC₅₀ values (μM) for the above compound were measured against the matrix metalloproteases indicated below, with the results indicated:

| | |
|---|---|
| MMP-1 | >100 |
| MMP-2 | >100 |
| MMP-3 | 0.16 |
| MMP-9 | >100 |
| MMP-12 | 3.4 |
| MMP-13 | 0.0012 |
| MMP-14 | >100 |

### EXAMPLE 4

3-Benzyl-2,4-dioxo-1,2,3,4-tetrahydro-quinazoline-6-carboxylic acid benzyl ester

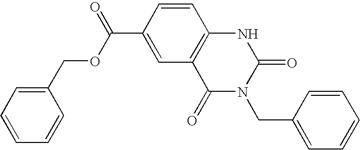

A mixture of 0.5 g (1.7 mmol) of the compound of 3-benzyl-2,4-dioxo-1,2,3,4-tetrahydroquinazoline-6-carboxylate, 0.44 g (1.7 mmol) of triphenylphosphine and 0.44 ml (4.3 mmol) of benzyl alcohol is stirred in 20 ml of THF. A solution of 0.27 ml (1.7 mmol) of DEAD in 10 ml of THF is added dropwise with stirring. Stirring is continued overnight at room temperature. The precipitate formed is filtered through Celite and the filtrate is concentrated under vacuum. The residue is dissolved in 50 ml of ethyl acetate and washed successively with water and then with saturated NaCl solution. After drying over MgSO₄ and concentration under vacuum, the crude product obtained is purified by flash chromatography on silica, eluting with a 50/50 mixture of hexane/EtOAc. The desired fractions are combined and the solvent is removed under vacuum to provide 0.190 g (yield=29%) of the desired crystalline compound.

MS: m/z 387.2 (M+H)+

NMR: DMSO ¹H δ (ppm): 5.06 (s,2H); 5.34 (s,2H); 7.22-7.46 (m, 10H); 8.20 (d,1H); 8.48 (s,1H); 11.89 (s,1H)

Activities measured by TPL assays for the various MMPs were as follows:

| | |
|---|---|
| MMP-1 | >30 |
| MMP-2 | >100 |
| MMP-3 | >30 |
| MMP-7 | >30 |
| MMP-9 | >100 |
| MMP-13 | 0.0295 |
| MMP-14 | >100 |

### EXAMPLE 5

### PD0331224

4-({[4-(4-Trifluoromethoxyphenyl)thien-2-yl]carboxamido} methyl) benzoic acid

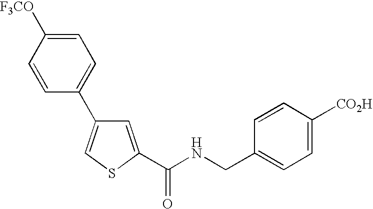

Step 1: 4-Bromothiophene-2-carbonyl chloride

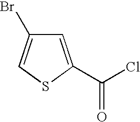

To a solution of 2.06 g of 4-bromothiophene-2-carboxylic acid in 60 ml of dichloromethane were added a few drops of dimethylformamide followed by the addition of 9.94 ml (2 equivalents) of oxalyl chloride. After stirring at room temperature for 2 hours, the mixture was concentrated under reduced pressure to afford 2.24 g of yellow oil, which was used crude in the next step.

Step 2: Ethyl 4-([(4-Bromothien-2-yl)carboxamidolmethyl]benzoate

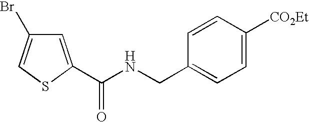

To a solution of 1.12 g of the compound obtained in the preceding step in 10 ml of dichloromethane were added 1.06 g (2 eq) of ethyl 4-(aminomethyl)benzoate and 1.04 ml of triethylamine (1.5 eq). The reaction mixture was stirred at room temperature for 17 hours. 50 ml of Water was then added and the solution was successively washed with 1.0N HCl and a saturated aqueous solution of sodium hydrogenocarbonate. The organic layer was dried over Na₂SO₄ and concentrated. The residue was purified on a silica gel column with 72:25 cyclohexane:ethyl acetate to yield 0.76 g (44%) of the desired compound.

N.M.R 1H (CDCl₃) δ (Ppm): 8.0 (m, 2H), 7.4 (m, 4H), 6.4 (bs, 1H), 4.6 (d, 2H), 4.3 (q, 2H), 1.40 (t, 3H).

Step 3: Ethyl 4-({[4-(4-trifluoromethoxyphenyl)thien-2-yl]carboxamido)methyl) benzoate

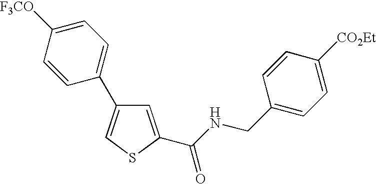

100 mg of the compound obtained in the preceding Step was dissolved in 5 ml of degassed DME. 61.5 mg of 4-(Trifluoromethoxy)phenyl-boronic acid (1.1 equivalents), 9.4 mg of tetrakis(triphenylphosphine)palladium(0) (0.03 equivalents) and 0.28 ml of a 2M potassium phosphate aqueous solution (2.1 equivalents) were then added under nitrogen atmosphere. The reaction mixture was heated to 80° C. for 3 hours, then diluted with 30 ml of ethyl acetate, washed with 2×15 ml of water, dried over sodium sulfate and concentrated. The residue was purified on a silica gel column using 7:3 cyclohexane:ethyl acetate to yield 60 mg (60%) of the desired compound.

MS (ES+) MHz 450 (M+1)

Step 4: 4-([[4-(4-Trifluoromethoxyphenyl)thien-2-yl]carboxamido}methyl)benzoic acid

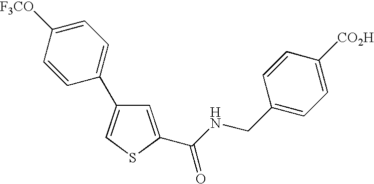

To a suspension of 60 mg of the compound obtained in the preceding step in 10 ml of 1:1 ethanol:water were added 16 mg (5 equivalents) of lithium hydroxide. The reaction mixture was stirred at room temperature for 17 hours then concentrated under reduced pressure. The aqueous remaining layer was acidified with 1N hydrochloric acid aqueous solution. After extraction with ethyl acetate, the organic layer was dried over sodium sulfate and concentrated to yield 49.5 mg (88%) of the desired compound as a white powder.

N.M.R ¹H (DMSO) δ (ppm): 9.2 (bs, 1H), 8.3 (s, 1H), 8.2 (s, 1H), 7.85 (m, 2H), 7.7 (m, 2H), 7.4 (m, 4H), 4.5 (d, 2H); MS (ES−) MHz 420 (M−1); Purity (HPLC): 100%

IC₅₀ values (μM) for the above compound were measured by TPL assays against the matrix metalloproteases indicated below, with the results indicated:

| | | |
|---|---|---|
| MMP-1 | >30 | |
| MMP-2 | >30 | |
| MMP-3 | >30 | |
| MMP-7 | >30 | |
| MMP-8 | >30 | |
| MMP-9 | >30 | |
| MMP-12 | >0.4 | MMP12 (+AH) 0.012 |
| MMP-13 | 0.00014 | MMP13 (+AH) 0.063 |
| MMP-14 | >30 | |

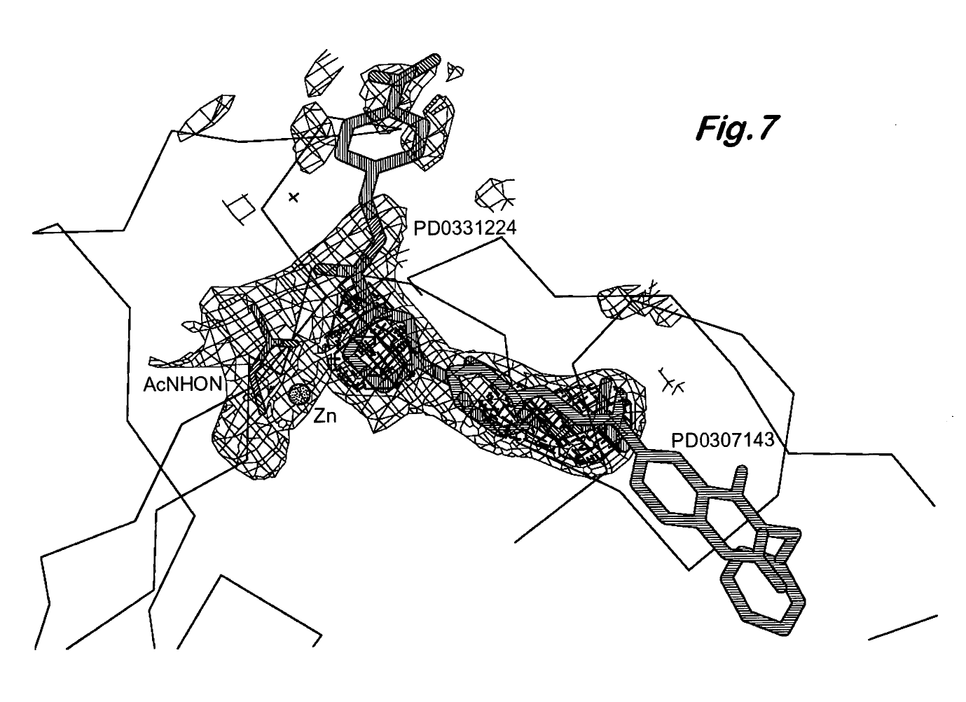

Crystallization conditions for complexes of MMP13 CD (Catalytic Domain) and the allosteric inhibitors PD0331224 and PD0307143—FIG. 7:

Recombinant human MMP 13 catalytic domain (CD) was used at 5-12 mg/ml in 50 mM Tris, pH 7.6, 0.02 nM Zn₂Cl, 10 mM Ca₂Cl.

Ternary complexes of MMP13 CD with inhibitors and acetohydroxamic acid were obtained by co-crystallization. The inhibitors were dissolved in DMSO (the final concentration of DMSO after mixing with protein did not exceed 5%) and mixed with diluted protein solution in at least 5:1 molar ratio. 100 mM of acetohydroxamic acid solution was added to the complex followed by incubation at 4° C. for four hours and centrifugation for 5 minutes at 3000 g. The supernatant was concentrated to 7-20 mg/ml. Crystallization was carried out in 2-4 μl hanging drops (1:1 ratio of complex to reservoir solutions) equilibrated against 0.5 ml of the reservoir solution. Complexes with inhibitors were crystallized using 18-22% PEG MME 5K, 0.2 M Li₂SO₄, 0.1 M HEPES pH 7.0 as reservoir solution.

### EXAMPLE 6

### [PF-0356231]—FIG. 10

(2?)-3-({[4-1(pyridin-4-yl)phényl]-thièn-2-yl]carboxamido)(phényl)propanoic acid hydrochloride [Alternatively 3-Phenyl-3-{[4-(4-pyridin-4-yl-phenyl)-thiophene-2-carbonyl]-amino}-propionic acid hydrochloride]

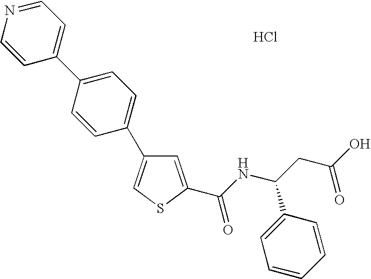

Step 1: Methyl 4-(4-nitrophenyl)thiophen-2-carboxylate

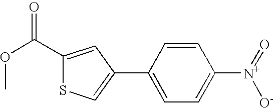

To a solution of methyl 4-bromothiophen-2-carboxylate and 1.2 equivalents of (4-nitrophenyl)boronic acid in DME were added 2.1 equivalents of a 2.0 M solution of potassium phosphate and 0.03 equivalent of tetrakis(triphenylphosphie)palladium (0). After stirring at 80° C. for 3 hours the mixture was concentrated under reduced pressure. The residue was diluted with ethyl acetate, filtered over Celite, washed with water, dried over sodium sulfate, and then concentrated under vacuum. The residue was purified on a silica gel column using 9:1 cyclohexane:ethyl acetate to yield 1.94 g (78%) of the desired compound.

Step 2: Methyl 4-(4-aminophenyl)thiophen-2-carboxylate

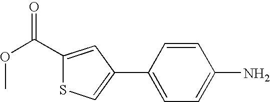

A solution of 1.94 g of the compound obtained in the preceding step and 194 mg of Pd (C) 10% in 20 ml of methanol was stirred at 50° C. over 6 hours under 10 bars of hydrogen. The mixture was then filtered over Celite and concentrated under vaccum to yield 1.51 g (88%) of the desired compound.

Step 3: Methyl 4-(4-bromophenyl)thiophen-2-carboxylate

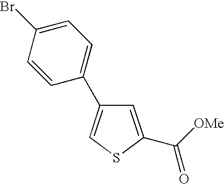

To a solution of 103 mg of the compound obtained in the preceding step in 1.5 ml of water and 0.6 ml of concentrated hydrobromic acid, cooled to 0° C. was added a solution of 35.5 mg of sodium nitrite (1.1 equivalents) in 0.5 ml of water. After stirring at 0° C. for 1 hour, a solution of 68 mg of copper bromide in 0.5 ml of concentrated hydrobromic acid was added drop-wise. The mixture was then stirred at 0° C. for 1 hour, diluted with ethyl acetate (30 ml), washed successively with water (3×15 ml), saturated solution of sodium bicarbonate (15 ml), and then water (15 ml). The organic layer was dried over sodium sulfate, filtered and concentrated under vacuum. The residue was purified on a silica gel column using 95:5 cyclohexane:ethyl acetate to yield 52 mg (40%) of the desired compound.

Step 4: Methyl 4-[4-(pyridin-4-yl)phenyl]thiophen-2-carboxylate

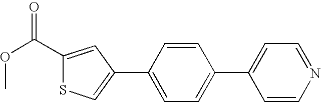

Methyl 4-(4-bromophenyl) thiophene-2-carboxylate (46 g, 0.16 mol, 1.0 wt), pyridylboronic acid (23 g, 0.19 mol, 0.5 wt) and dimethoxyethane (1.3 L, 30.0 vol) were charged to the reaction vessel under an atmosphere of nitrogen. The reaction vessel was purged with nitrogen three times. Tetrakis(triphenylphoshine)palladium(0) (5.3 g, 0.005 mol, 0.12 wt) and aqueous tripotassium phosphate (2M, 69 g in 160 ml water, 3.5 vol) were charged and the vessel was purged three more times with nitrogen. The solution was heated to 80° C. and maintained at this temperature for 48 hours and allowed to cool to room temperature overnight. Ethyl acetate (2.0 L, 10.7 vol) and water (0.2 L, 1.1 vol) was added and the layers separated. The organic layer was washed with water (1.0 L, 10.7 vol) and filtered through glass microfibre. Hydrochloric acid (2M, 0.2 L, 0.4 mol, 8.7 vol) was added with stirring and the yellow precipitate was filtered through glass microfibre filter. The solid was washed with water (1.1 L, 23.9 vol) and dried by suction for 1 hour. The water-wet solid (60 g) was used in the next step without any further drying or purification.

N.M.R. ¹H (DMSO) δ (ppm): 8.91 (d, 2H), 8.50 (s, 1H), 8.38 (s, 1H), 8.36 (d, 2H), 8.10 (d, 2H), 8.06 (d, 2H), 3.45 (s, 3H).

Step 5: 4-[4-(pyridin-4-yl)phenyl]thiophen-2-carboxylic acid

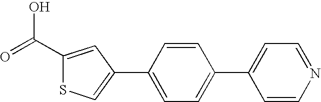

Methyl 4-(4-pyridin-4-ylphenyl) thiophene-2-carboxylate (60 g wet, ≦0.16 mol) obtained in the preceding Step, lithium hydroxide monohydrate (20 g, 0.5 mol), methanol (400 ml) and water (100 ml) were charged to the reaction vessel to give a fine white suspension. The reaction mixture was heated to 50° C. and maintained at this temperature overnight. The methanol was removed in vacuo and hydrochloric acid (4M, 180 ml, 0.72 mol) was added slowly. The solid was filtered through glass microfibre filter, washed with water (50 ml) and dried by suction overnight. The wet solid was slurried in THF (1.0 L) for 1 hour and filtered. The yellow solid was ground to a fine powder and dried over phosphorus pentoxide at 40° C., under vacuum for 10 days to give the desired product 19.4 g (44.5% over steps 4 and 5), after which Karl-Fisher analysis gave a water content of 34% w/w.

N.M.R. ¹H (DMSO) δ (ppm): 8.88 (d, 2H), 8.43 (s, 1H), 8.30 (d, 2H), 8.28 (s, 1H), 8.08 (d, 2H), 8.02 (d, 2H)

Step 6: Ethyl (2R)-3-([4-[4-(Pyridin-4-yl)phenyl]thien-2-yl]carboxamido)(phenyl) propanoate

To a solution of the compound obtained in the preceding step 5 in 6 ml of dimethylformamide were added 1.1 equivalents of ethyl (2R)-amino(phenyl) propanoate, 1.1 equivalents of O-(7-azabenzotriazol-1-yl)-N,N,N′,N′-tetramethyluronium hexafluorophosphate and 0.1 equivalent of diisopropylethylamine. After 17 hours of stirring at room temperature, the mixture was hydrolyzed. The precipitate was filtered, washed with water and dried to yield 1.43 g (100%) of the desired compound.

N.M.R. ¹H (DMSO) δ (ppm): 8.15 (d, 1H), 8.76 (d, 2H), 8.35 (s, 1H), 8.22 (s, 1H), 7.95 (d, 2H), 7.90 (d, 2H), 7.88 (d, 2H), 7.44 (d, 2H), 7.35 (t, 2H), 7.26 (t, 1H), 5.42 (q, 1H), 4.05 (q, 2H), 2.95 (m, 2H), 1.10 (t, 3H); Purity (HPLC): 100%

Step 7: (2R)-3-({4-[(pyridin-4-yl)phenyl]-thien-2-yl}carboxamido)(phenyl) propanoic acid hydrochloride

The above compound (0.99 g; yield:68%) was obtained following the process described in the Step 3 of Example 5 and using as substrate the compound obtained in the preceding step 6.

N.M.R. ¹H (DMSO) δ (ppm): 9.02 (d, 1H), 8.88 (d, 2H), 8.46 (s, 1H), 8.34 (d, 2H), 8.30 (s, 1H), 8.11 (d, 2H), 7.95 (d, 2H), 7.44 (d, 2H), 7.35 (t, 2H), 7.26 (t, 1H), 5.43 (q, 1H), 2.89 (m, 2H); Purity (HPLC): 100%

IC₅₀ values (μM) for the above compound were measured by TPL assays against the matrix metalloproteases indicated below, with the results indicated:

| | | |
|---|---|---|
| MMP-1 | >101 | |
| MMP-2 | >0.39 | |
| MMP-3 | >1.7 | |
| MMP-8 | >0.98 | |
| MMP-9 | >1.4 | |
| MMP-12 | 0.014 | MMP12 (+AH) 0.004 |
| MMP-13 | 0.27 | |

Crystallization with compound of example 6—FIG. 10:

A complex is obtained as the same manner than the complex with MMP13 using MMP12 CD and compound of Example 6. Complex with inhibitor is crystallized using 2.2-2.8 sodium chloride and 0.1 M imidazol pH 8.0, with a protein concentration at 9 mg/ml.

Structure of the Resulting Complex

The above compound binds deeply into the S1′ pocket of MMP12. The crystal structure shows clearly that the compound does not chelate the Zn atom and that it binds allosterically to MMP12 through the S₁′, or S1″, or S1′ and S1″ pocket(s). The following points concerning its mode of binding were found:

It makes two direct hydrogen bonds with the protein, as well as several hydrophobic contacts (FIG. 10).

The carbonyl oxygen 013 makes a hydrogen bond to the backbone NH of Leu164 (d[NH—O]=2.2A) and Ala165 (d[NH—O]=2.8A).

The amide NH make a hydrogen bond to the Pro221 carbonyl oxygen (d[O—HN]=1.9A).

The carboxylate group on R3 binds to solvent, and there is no hydrophobic contact between the cyclohexyl group and the protein.

The thiazol and phenyl aromatic rings of the core structure make hydrophobic contacts with Val 198 (d=3.8A), His201 and the α- and β-methylene groups of Tyr223 (d=3.3A and 3.9A respectively).

The 4-pyridyl group makes hydrophobic contacts with Leu197 (d=3.7A) and Leu218 (d=3.7).

Acetohydroxamate binds to the Zinc ion with both its oxygen atoms, and makes two hydrogen bonds to Glu202 and Ala165 with its OH and NH respectively.

### EXAMPLE 7

### [CP271485]—FIG. 11

4-Benzyl-6-(1-methyl-2,2-dioxo-2,3-dihydro-1H-2λ⁶-benzo[c]isothiazol-5-yl)morpholin-3-one

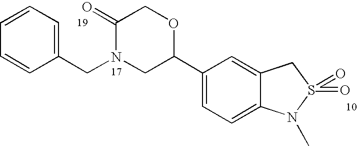

This compound was obtained using the process described in the patent U.S. Pat. No. 3,704,299. IC₅₀ values (μM) for the above compound were measured by TPL assays against the matrix metalloproteases indicated below, with the results indicated:

| | | |
|---|---|---|
| MMP-12 | 17 | MMP12 (+AH) 0.27 |
| MMP-13 | 3.6 | |

Crystallization with Compound of Example 7—FIG. 11:

A complex is obtained as the same manner than the complex with MMP13 using MMP12 CD and compound of Example 7. Complex with inhibitor is crystallized using 0.1 M sodium Cacodylate pH 6.5 and 1.0-1.4 M sodium acetate, with a protein concentration at 10 mg/ml.

Binding of the Compound to Form the Complex

The above compound binds deeply into the S1′ pocket of MMP12. The crystal structures show clearly that it does not chelate the Zn atom and that it binds allosterically MMP12 through the S1′, or S1″, or S1′ and S1″ pocket(s). The following points were noted:

It makes three direct hydrogen bonds with the protein, as well as several hydrophobic contacts and one indirect hydrogen bond with the protein via water molecules.

The carbonyl oxygen O19 makes an hydrogen bond with the backbone NH of Ala182 (d_{[NH—O]}=2.6A) and Leul81 (d_{[NH—O]}=1.8A).

The sulfonamide β-oxygen O10 makes an hydrogen bond with the backbone NH of Lys241 (d_{[NH—O]}=2.4A), and binds to the carbonyl oxygen of Lys241 via Wat8 (d_{[O—O}]=2.9A).

The phenyl fragment of the benzyl group attached to N17 makes weak hydrophobic contacts with Ile180 sidechain (d=4.2A).

The amide π-electrons make hydrophobic contacts with Leu181 (d=3.8A).

The core structure phenyl ring makes hydrophobic contacts with Tyr240 α- and β-methylene groups (d=3.9 and 4.2A respectively) and His218 (d=3.6).

The N-methyl group makes hydrophobic contacts with Leu214 (d=3.8A) and Val235 (d=3.9A). Acetohydroxamate binds to the Zinc ion with both its oxygen atoms, and makes two hydrogen bonds to Glu219 and Leu181 with its OH and NH respectively.

### EXAMPLE 8

### [0342938]—FIG. 12

Trans 3-[4-(4-pyridyl)phenyl]-5-isothiazole carboxamido methyl-cyclohexane carboxylic acid, Potassium Salt

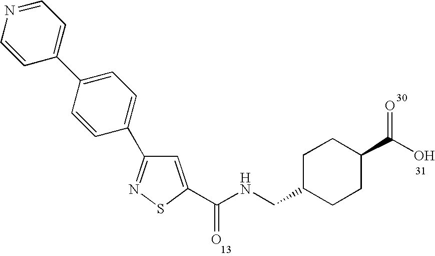

Step 1: 4-bromo-α-(p-toluenesulfonyloxyimino)benzylcyanide

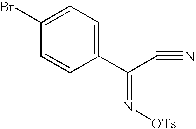

The above compound was prepared as described in WO 4346094 using 4-bromobenzylcyanide as substrate (4.8 g; yield: 26%).

N.M.R. ¹H (DMSO) δ (ppm): 8 (d, 2H), 7.85 (d, 2H), 7.72 (d, 2H), 7.6 (d, 2H), 2.5 (s, 3H); Purity (HPLC): 83.37%

Step 2: Methyl 3-(4-bromophenyl)-4-amino-5-isothiazolecarboxylate

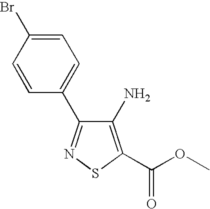

The desired product is obtained following the process described in WO 4346094 (2.66 g; yield: 66%).

MS (ES+) M/z 313 (M+1); Purity (HPLC): 89.64%

Step 3: Methyl 3-(4-bromophenyl)-5-isothiazolecarboxylate

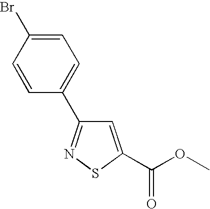

The desired product was obtained following the process described in WO 4346094 (2.8 g; yield: 100%).

MS (ES+) MHz 299 (M+1); Purity (HPLC): 85.15%

Step 4: 3-(4-Bromophenyl)-5-isothiazole carboxylic acid

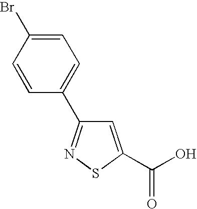

To a solution of 2.79 g of compound obtained in the preceding step in 45 ml of methanol were added 1.6 g (3 equivalents) of sodium hydroxide. The reaction mixture was heated to reflux over 2.5 hours, after which it was poured onto a mixture of ice and water and acidified with 1N hydrochloric acid aqueous solution. The resulting precipitate was filtered off, rinced with water and ethyl ether and dried to yield 2 g (81%) of the desired compound.

MS (ES−) M/z 283 (M−1); Purity (HPLC): 80%

Step 5: Ethyl trans 3-(4-bromophenyl)-5-isothiazolecarboxamidomethyl-cyclohexane carboxylate

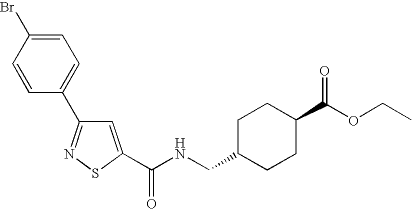

To a solution of 1 g of the compound obtained in step 4 in 35 ml of dimethylformamide were added 0.65 g of ethyl trans 4-(aminomethyl)cyclohexanecarboxylate (prepared by esterification of the commercially available corresponding acid) and 1.34 g of O-(7-azabenzotriazol-1-yl)-N,N,N′,N′-tetramethyluronium hexafluorophosphate. The reaction mixture was cooled to 0° C. and 1.22 ml of diisopropylethylamine were added dropwise. After stirring at room temperature overnight, the mixture was hydrolyzed and then extracted with dichloromethane. The combined organic layer was then dried over sodium sulfate and concentrated. The residue was purified on a silica gel column using 95:5 dichloromethane:ethanol to yield 0.4 g (28%) of the desired compound.

N.M.R. ¹H (DMSO) δ (ppm): 8.85 (t, 1H), 8.45 (s, 1H), 7.9 (d, 2H), 7.75 (d, 2H), 4.05 (q, 2H), 3.15 (t, 2H), 2.25 (t, 1H), 1.925 (d, 2H), 1.8 (d, 2H), 1.55 (m, 1H), 1.3 (q, 2H), 1.2 (t, 3H), 1.05 (q, 2H); MS (ES+) M/z 452 (M+1); Purity (HPLC): 90.18%

Step 6: Ethyl trans 3-[4-(4-pyridyl)phenyl]-5-isothiazole-carboxamidomethyl-cyclohexane carboxylate

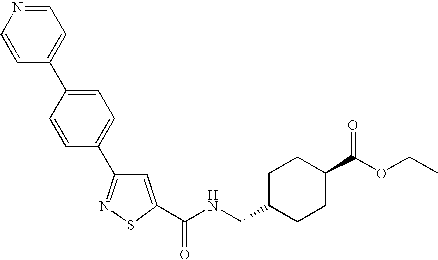

400 mg of the compound obtained in the preceding step was dissolved in 25 ml of degassed DME. 1.2 Equivalents of 4-pyridyl boronic acid, 0.03 equivalents of tetrakis(triphenylphosphine)palladium(0) and 2.1 equivalents of a 2M potassium phosphate aqueous solution were then added under nitrogen atmosphere. The reaction mixture was heated at 80° C. for 24 hours, then cooled and diluted with 30 ml of ethyl acetate, washed with water, dried over sodium sulfate and concentrated. The residue was purified on a silica gel column using 98:2 dichloromethane:methanol to yield 46 mg (11%) of the desired compound.

MS (ES+) MHz 450 (M+1); Purity (HPLC): 100%

Step 7: trans 3-(4-bromophenyl)-5-isothiazolecarboxamidomethyl-cyclohexane carboxylatic acid, Potassium Salt

To a solution of 46 mg of the compound obtained in the preceding Step in 2 ml of ethanol were added 12 mg (2 equivalents) of potassium hydroxide. The mixture was stirred at room temperature overnight. The precipitate obtained was then filtered, washed with water, and dried to yield 18 mg (39%) of the desired compound.

N.M.R. ¹H (DMSO) δ (ppm): 9.17 (m, 1H), 8.75 (s, 1H), 8.67 (d, 2H), 8.19 (d, 2H), 7.99 (d, 2H), 7.81 (d, 2H), 3, 11 (t, 2H), 1.77 (m, 4H), 1.67 (m, 1H), 1.53 (m, 1H), 1.20 (m, 2H), 0.87 (q, 2H); MS (ES+) M/z 422 (M+1); Purity (HPLC): 96.6%

IC₅₀ values (μM) for the above compound were measured by TPL assays against the matrix metalloproteases indicated below, with the results indicated:

| | | |
|---|---|---|
| MMP-12 | 0.57 | MMP12 (+AH) 0.015 |
| MMP-13 | 1.9 | |

Crystallization conditions for complexes of MMP13 CD (Catalytic Domain) and inhibitors of MMP12—FIG. 12:

A complex is obtained as the same manner than the complex with MMP 13 using MMP12 CD and compound of example 8. Complex with inhibitor is crystallized using 10% (w/w) of PEG 8K and 9% (w/w) of PEG 1K.

Structure of the complex with MMP12

The above compound binds deeply into the S1′ pocket of MMP13. The crystal structures show clearly that it does not chelate the Zn atom and that 12 binds allosterically to MMP12 through the S1′, or S1″, or S1′ and S1″ pocket(s). The following points were noted:

It makes three direct hydrogen bonds with the protein, as well as several hydrophobic contacts and one indirect hydrogen bond with the protein via water molecules.

The carbonyl oxygen 013 makes an hydrogen bond to the backbone NH of Leu181 (d_{[NH—O]}=1.7A) and NH of Ala182 (d[NH-o]=2.6A).

The carboxylate oxygen 030 makes a hydrogen bond with the backbone NH of Tyr240(d_{[NH—O]}=2.5A), whereas the other carboxylate hydrogen (031) binds to Gly179 carbonyl oxygen via Wat61(d_{[O—O]}=1.8A).

The phenyl group on R3 makes hydrophobic contacts with Ile180 sidechain (d=3.2A).

The sidechain amide π-electrons makes hydrophobic contacts with Leu181 (d=4.0A).

The core structure thiophene and phenyl aromatic rings make hydrophobic contacts with Thr215 (d=3.8A), Tyr240 α-methylene (d=3.8A) and His218 (d=3.6A), as depicted in FIG. 12.

The R1 4-pyridyl group makes hydrophobic contacts with Lys241 γ- and ε-methylene groups (d=3.9 and 3.4A respectively) on the upper side, as well as with Val235 (d=3.5A) on the lower side.

Acetohydroxamate binds to the Zinc ion with both its oxygen atoms, and makes two hydrogen bonds with Glu219 and Leu181 with its OH and NH respectively.

Appendix 1

Tests for Allosteric Binding

“Allosteric MMP inhibitor” refers to a compound, irrespective of its nature, that is able to decrease or inhibit the action of a targeted MMP or small numbers of MMPs by binding to a site different from the active catalytic site of said targeted MMP(s). Preferably, this term refers to a compound that does not bind to the functional metal cation of the catalytic site of the targeted MMP enzyme(s). Such allosteric MMP inhibitors may be either mixed or uncompetitive inhibitors with respect to cation ligands. Cation ligand include any molecule able to bind to the catalytic cation like for example enzyme substrates or any cation chelator.

“Mixed inhibitor” refers to an inhibitor binding to the enzyme, to an enzyme—substrate complex, or to an enzyme-product complex. A mixed MMP inhibitor does not compete with a ligand to the functional metal cation for binding to said cation

“Uncompetitive inhibitor” refers to an inhibitor binding to the enzyme-cation ligand complex only. An uncompetitive MMP inhibitor does not compete with the ligand to the functional metal cation for binding to the functional metal cation of the metalloenzyme-substrate complex.

Screening methods that discriminate between competitive inhibition binding to the cation in the MMP active site and inhibition without binding to said cation (i.e. mixed or uncompetitive inhibitors) are therefore required.

In such screening methods, the IC50 of putative allosteric MMP inhibitors are compared in the presence or absence of a ligand that chelates readily to zinc but is a sufficiently small molecule that it does not substantially interfere with the binding of the inhibitor to its intended location adjacent to the catalytic domain. Although ligand size is not critical, a bulky ligand will block more binding sites of the MMP than a smaller ligand, and may thus prevent potential inhibitors that bind close to, but not at, the functional metal cation from being identified. Suitable ligands are therefore low molecular weight molecules, e.g. of molecular weight 30-750 Daltons, preferably 40-500 Daltons and most preferably 50-250 Daltons. A preferred ligand is acetohydroxamic acid (CH₃CONHOH, AcNHOH, or AcN(H)OH). Other ligands that may be used include acetic acid, propanoic acid, N-hydroxy-propanamide, acetoacetic acid, malonic acid, ethanethiol, 1,3-propanedithiol, N-hydroxy-benzamide, imidazole, 2-mercaptoethanol, cyanide, thiocyanate, 2,4,6-trihydroxypyrimidine, 1,10-phenanthroline, and the like. Low molecular weight, known inhibitors of a particular MMP such as, for example, a dipeptide inhibitor may be used as the ligand to the functional metal cation.

Briefly, in such screening methods, the ability of a compound to inhibit MMP in the absence of a ligand to the functional metal cation, is compared to its ability to inhibit the same MMP in the presence of said ligand to the functional metal cation. This comparison can be, for example, by calculating a ratio of inhibition.

One method of comparing is to determine, by conventional means, the inhibition as IC50 values, respectively, and to calculate an IC50 value ratio as the IC50 value of the compound with the MMP in the presence of the ligand (at a well defined concentration) divided by the IC50 value of the compound in the absence of the ligand. If the IC50 value ratio is lower or equal to 1, the inhibitor is mixed or uncompetitive, and the inhibitor is synergistic with the ligand to the functional metal. On the other hand, if the ratio is >1, the inhibitor is competitive, mixed or uncompetitive.

There are many other ways of determining the inhibition ratio such as, for example, by dividing the inhibition of a compound, at a known concentration, in the presence of the ligand by the inhibition of the compound, at the same concentration, in the absence of the ligand. Another example of determining the inhibition ratio is dividing the percent inhibition of a compound at a known concentration in the presence of the ligand by the percent inhibition of the compound at the same concentration in the absence of the ligand. Enzyme kinetics experiments may be further employed, when necessary, to differentiate between competitive, mixed, and uncompetitive inhibitors.

In practice, an assay for a MMP may be carried out with and without a test compound, and the rates of hydrolysis, as indicated by the steady-state initial reaction velocities of cleavage of substrate by the MMP, may be measured to determine inhibitory activity of the test compound. Initial reaction velocities of cleavage of substrate by the MMP may be determined by measuring the rate of substrate cleavage or the rate of reaction product formation. Measurements may be made utilizing spectrophotometric means, fluorimetric means, or by SDS-polyacrylamide gel electrophoresis (“SDS-PAGE”).

For example, when fluorimetric means are utilized, changes in absorbance without a test compound and with test compound at different concentrations of compound such as, for example, 100, 10, and 1 μM, or 100, 10, and 1 nM may be measured. Alternatively when fluorimetric means are utilized, changes in fluorescence may be measured by comparing fluorescence without a test compound to fluorescence with a test compound, wherein the comparisons are performed at different concentrations of test compound, such as those described immediately above. The compound concentration is then plotted on the X-axis against the percentage of control activity observed for experiments with compound versus experiments without compound (i.e., (velocity with a ligand to the functional metal cation) divided by (velocity without a ligand to the functional metal cation)×100) on the Y-axis to define IC50 values. These experiments are run both in the presence and the absence of the ligand to the functional metal cation. Data are fit to the equation:

% control activity=100/[I+(([I]/IC50)slope)]

where [I] is the compound concentration, IC50 is the concentration of compound where the reaction rate is 50% inhibited relative to the control reaction, and slope is the slope of the IC50 curve at the curve's inflection point, using nonlinear least-squares curve fitting equation regression.

Alternatively, the inhibition ratio may be determined by comparing the amount of substrate cleavage or reaction product formation at a single time point (e.g., 30 minutes after addition of compound), measured by spectrophotometric means, fluorimetric means, or SDS-PAGE, to the initial amount of substrate or reaction product for a compound in the presence of a ligand to the functional metal cation, and then in the absence of a ligand to the functional metal cation.

The assay method, and substrate employed therein, for a particular MMP is a method and substrate known in the art to be useful for screening for inhibitors of said MMP. A ligand to the functional metal cation of the MMP is first identified by screening compounds for MMP inhibition using conventional means. Preferably, the Ki of the ligand with the metalloenzyme is determined by conventional means, and the concentration of ligand employed in the invention method is about equal to the Ki value, or within the range of Kd as described below.

Such ligands to the functional metal cation are any compound that binds to the functional metal cation of an MMP in the context of the present invention. The binding may be of a direct or indirect character. Indirect binding of a ligand via a noncovalent bond to the functional metal cation includes coordination via a bridging water molecule or conjugate acid/base thereof.

In principle, a ligand of any potency, concentration, or size will work in the screening method provided that the ligand is at a concentration such that, in the assay mixture, some unbound MMP is present and some bound MMP is present such that catalytic activity of the enzyme is detectable by the assay method employed, and the graded inhibition of the enzyme activity, in the presence or absence of the ligand, is differentiable.

Preferred is a concentration of the ligand of from about 0.5 to about 3 Kd, wherein “Kd” is the disassociation constant for the ligand-enzyme complex. One Kd is equal to the Ki of the ligand with the enzyme. More preferred is a concentration of the ligand that is from about 1 Kd to about 2 Kd. A concentration of the ligand that is about 1 Kd means that about 50% of the target enzyme is bound to the ligand and about 50% of the target enzyme is free (i.e., unbound). In any of the embodiments of the instant invention described above, preferred is a concentration of the ligand that provides from about 20% bound target enzyme to about 90% bound target enzyme. More preferred is a concentration of the ligand that provides from about 35% bound target enzyme to about 90% bound target enzyme. Still more preferred is a concentration of the ligand that provides from about 40% bound target enzyme to about 90% bound target enzyme.

An alternative preferred approach is to test in the presence of a concentration of the chelator that is about 1 Kd in order to minimize the risk of eliminating potentially useful compounds. In that approach, screening at the Kd of acetohydroxamate(AH) identifies statistically most of the potentially useful compounds whatever their mechanism of action is, after which these compounds can be further studied and the ones with ratio>1 deconvoluted to discriminate between which ones are competitors of AH, and which ones are mixed.

Screening methods to identify noncompetitive and uncompetitive MMP inhibitors are further detailed in examples 1-6 using either full-length MMP or catalytic domains of MMPs.

### BINDING EXAMPLE 1

Activation of Pro-MMPs

Pro-MMPs produces as latent or inactive zymogens can be activated by autoactivation and by the action of other MMPs or proteases such as furin, plasmin, and trypsin, as well as by organomercurial compounds. For example, p-amino phenylmercuric acetate (APMA) or other types of organomercurials such as p-(hydroxymercuri) benzoate (PHMB), phenylmercuric chloride (PMC) and mersalyl may be used as described in the activation protocol from Strickin et al. (1983) Biochemistry 22: 61.

Organomercurial Stock Solutions:

Prepare stock solutions in 0.1 M NaOH at a concentration range of 10-50 mM just prior to use. Although not absolutely necessary, the stock solution may be adjusted to pH 11 with 5 N HCl (Marcy et al. (1991) Biochemistry 30: 6476).

Pro-MMPs

Prepare proenzyme solutions in 0.1 M Tris-HCl, PH 7.5 at 1-2 mg/ml.

Activation of Pro-MMPs:

To initiate activation, combine pro-MMP solution with organomercurial stock in a 10:1 volume ratio. Incubate the mixture at 37° C. for 2-3 hours.

When more organomercurial is desired, make the stock solution more concentrated. Do not exceed the above ratio so that the volume of organomercurial in the mixture is more than about 1/10 of that of the pro-MMP solution. It is recommended that an analytical run be conducted first to determine the optimal incubation time. For example, a small-scale experiment with fixed concentration of proMMP and organomercurial would be incubated as described above. Remove aliquots of sample at various time points. Stop the reaction by the addition of concentrated SDS-sample buffer (for example add 10 μl of 2× sample buffer to a 10 μl aiquot or add 2 μl of 5× sample buffer to an 8 μl aliquot) and then boil. The progress of activation can then be monitored qualitatively by analysing these time aliquots on a 12% SDS-PAGE gel.

Removing Organomercurial from the Mixture After Activation:

The activated MMPs can be used without the removal of the organomercurial. However, if desired, organomercurial may be removed by gel filtration (Marcy et al (1991) supra).

### BINDING EXAMPLE 2

Fluorigenic Peptide-1 Substrate Based Assay for Identifying Competitive, Noncompetitive, or Uncompetitive Inhibitors of MMP-13:

The catalytic domain of the MMP-13 enzyme, namely matrix metalloproteinase-13 catalytic domain (“MMP-13CD”), rather than the corresponding full-length enzyme, MMP-13 was used in such assay. It has been shown previously by Ye Qi-Zhuang, Hupe D., and Johnson L. (Current Medicinal Chemistry, 1996;3:407-418) that inhibitor activity against a catalytic domain of an MMP is predictive of the inhibitor activity against the respective full-length MMP enzyme.

Final Assay Conditions:

50 mM HEPES buffer (pH 7.0)

10 mM CaCl₂

10 μM fluorigenic peptide-1 (“FP 1”) substrate

0 or 15 mM acetohydroxamic acid (AcNHOH)=1 Kd

2% DMSO (with or without inhibitor test compound)

0.5 nM MMP-13CD enzyme

Stock Solutions:

(1) 10× assay buffer: 500 mM HEPES buffer (pH 7.0) plus 100 mM CaCl₂

(2) 10 mM FP1 substrate: (Mca)-Pro-Leu-Gly-Leu-(Dnp)-Dpa-Ala-Arg-NH₂ (Bachem, M-1895; “A novel coumarin-labeled peptide for sensitive continuous assays of the matrix metalloproteinases,” Knight C. G., Willenbrock F., and Murphy, G., FEBS Lett., 1992;296:263-266). Prepared 10 mM stock by dissolving 5 mg FP1 in 0.457 mL DMSO.

(3) 3 M AcNHOH: Prepared by adding 4 mL H₂O and 1 mL 10× assay buffer to 2.25 g AcNHOH (Aldrich 15,903-4). Adjusted pH to 7.0 with NaOH. Diluted volume to 10 mL with H₂O. Final solution contained 3 M AcNHOH, 50 mM HEPES buffer (pH 7.0), and 10 mM CaCl₂.

(4) AcNHOH dilution buffer: 50 mM HEPES buffer (pH 7.0) plus 10 mM CaCl₂

(5) MMP-13CD enzyme: Stock concentration=250 nM.

(6) Enzyme dilution buffer: 50 mM HEPES buffer (pH 7.0), 10 mM CaCl₂, and 0.005% BRIJ 35 detergent (Calbiochem 203728; Protein Grade, 10%)

Procedure (for one 96-Well Microplate):

A. Prepared Assay Mixture:

1100 μL 10× assay buffer

11 μL 10 mM FP1

55 μL 3 M AcNHOH or 55 μL AcNHOH dilution buffer

8500 μL H₂O

B. Diluted MMP-13CD to 5 nM Working Stock:

22 μL MMP-13CD (250 nM)

1078 μL enzyme dilution buffer

C. Ran Kinetic Assay:

1. Dispensed 2 μL inhibitor test sample (in 100% DMSO) into well.

2. Added 88 μL assay mixture and mixed well, avoiding bubbles.

3. Initiated reactions with 10 μL of 5 nM MMP-13CD; mixed well, avoiding bubbles.

4. Immediately measured the kinetics of the reactions at room temperature. Fluorimeter: Fₘₐₓ Fluorescence Microplate Reader & SOFTMAX PRO Version 1.1 software (Molecular Devices Corporation; Sunnyvale, Calif. 94089).

Protocol menu:

| | |
|---|---|
| excitation: 320 nm | emission: 405 nm |
| run time: 15 min | interval: 29 sec |
| RFU min: −10 | RFU max: 200 |

Vₘₐₓ points: 32/32

D. Compared % of Control Activity and/or IC₅₀ with Inhibitor Test Compound AcNHOH Hydrolysis of the fluorigenic peptide-1 substrate, [(Mca)Pro-Leu-Gly-Leu-Dpa-Ala-Arg-NH₂; Bachem, catalog number M-1895], wherein “Mca” is (7-methoxy-coumarin-4-yl)acetyl and “Dpa” is (3-[2,4-dinitrophenyl]-L-2,3-diaminopropionyl), was used to screen for MMP-13 catalytic domain (CD) inhibitors. (Dpa may also be abbreviated as “Dnp”.) Reactions (100 pL) contained 0.05 M Hepes buffer (pH 7), 0.01 M calcium chloride, 0.005% polyoxyethylene (23) lauryl ether (“Brij 35”), 0 or 15 mM acetohydroxamic acid, 10 μM FP 1, and 0.1 mM to 0.5 nM inhibitor in DMSO (2% final).

After recombinant human MMP-13CD (0.5 nM final) was added to initiate the reaction, the initial velocity of FP1 hydrolysis was determined by monitoring the increase in fluorescence at 405 nm (upon excitation at 320 nm) continuously for up to 30 minutes on a microplate reader at room temperature. Alternatively, an endpoint read can also be used to determine reaction velocity provided the initial fluorescence of the solution, as recorded before addition of enzyme, is subtracted from the final fluorescence of the reaction mixture. The inhibitor was assayed at different concentration values, such as, for example, 100, 10, and 1 μM, or 100, 10, and 1 nM. Then the inhibitor concentration was plotted on the X-axis against the percentage of control activity observed for inhibited experiments versus uninhibited experiments (i.e., (velocity with inhibitor) divided by (velocity without inhibitor)×100) on the Y-axis to determine IC₅₀ values. This determination was done for experiments done in the presence, and experiments done in the absence, of acetohydroxamic acid. Data were fit to the equation: % control activity=100/[I+(([I]/IC₅₀)^{slope})], where [I] is the inhibitor concentration, IC₅₀ is the concentration of inhibitor where the reaction rate is 50% inhibited relative to the control, and slope is the slope of the IC₅₀ curve at the curve's inflection point, using nonlinear least-squares curve-fitting equation regression.

Results of Example 2 are shown below in Table 1 in the column labeled “IC₅₀ Ratio (±)”. In Table 1, noncompetitive or uncompetitive inhibitors that have an IC₅₀ Ratio (±) ratio of <1 are synergistic with AcNHOH, while competitive inhibitors have an IC₅₀ Ratio (±) of >1, unless otherwise indicated.

TABLE 1

| | | Without | With | IC₅₀ |
|---|---|---|---|---|
| Entry | | AcNHOH | AcNHOH | Ratio |
| No. | Compound Tested | IC₅₀ (μM) | IC₅₀ (μM) | (±) |
| 1 | 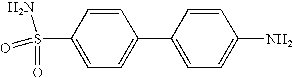 | 34 | 16 | 0.47^{a} |
| 2 | 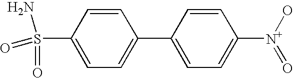 | 84 | 3.1 | 0.04 |
| 3 | 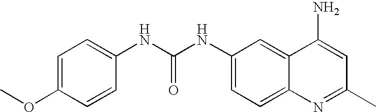 | 126 | 71 | 0.56 |
| 4 | 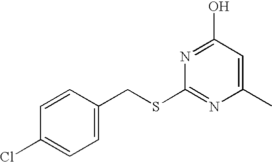 | 64 | 4.7 | 0.07^{a} |
| 5 | 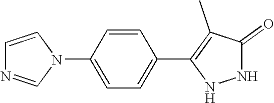 | 9.5 | 11 | 1.16 |
| 6 | 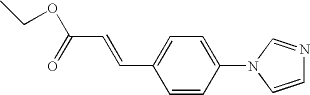 | 17 | 14 | 0.82 |
| 7 | 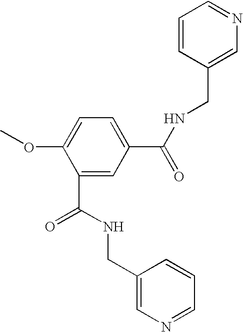 | 27 | 11 | 0.41 |
| 8 | 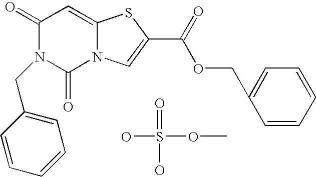 | 11 | 5.2 | 0.47 |
| 9 | 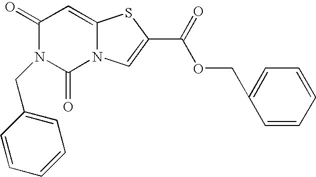 | 0.19 | 0.07 | 0.37^{a} |
| 10 | 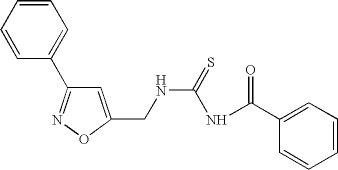 | 2.3 | 0.2 | 0.08 |

^{a}Kinetics experiments (not shown) demonstrate these compounds are noncompetitive inhibitors of MMP-13CD.

In a similar manner, an assay may be run wherein 1,10-phenanthroline is substituted for acetohydroxamic acid to identify a competitive, noncompetitive, or uncompetitive inhibitors of MMP-13CD.

### BINDING EXAMPLE 3

Fluorigenic Peptide-1 Substrate Based Assay for Identifying Competitive, Noncompetitive, or Uncompetitive Inhibitors of MMP-12

Materials and Methods

Enzyme and Reagents:

Human MMP-12 catalytic domain (“MMP-12CD”) was cloned, expressed in E. coli and purified using a denaturation/renaturation method. A fluorigenic petide-1 (FP-1) with the sequence: Mca-Pro-Leu-Gly-Leu-Dap(Dnp)-Ala-Arg-NH₂ was purchased from Bachem (ref: M-1895). Stock solution was prepared in DMSO at 10 mM and kept at −20° C. All the other reagents were from Sigma.

Plate Preparation:

For screening, 4 μL of the compounds were added to 384-well black microplates at 250 μM in 25% DMSO. For IC₅₀ determination, a range of 8 dilutions were prepared in 25% DMSO, and 4 μL of each concentration were added to the plates in duplicates.

Assay:

The reaction was started by sequential addition of 41 μL of the FP-1 (10 μM final concentration) in assay buffer (50 mM Tris-HCl, 10 mM CaCl₂) containing 5 mM AcNHOH and 5 μL of enzyme diluted in assay buffer containing 0.005% Brij-35. The microplates were incubated for 20 minutes at room temperature. The fluorescence changes were recorded in a Fluostar (BMG) instrument using excitation filter at 320 nm and emission filter at 405 nm. Results in wells containing compounds were calculated as a percentage of the fluorescence signal in control wells that received aqueous DMSO, but no compound (maximum signal), or no enzyme (minimum signal).

Results:

The signal to background ratio was between 5 to 8 with Z□ value (a quality control factor, see JI-HU ZHANG et. al., Journal of Biomolecular Screening, 1999;4(2):67-73) in screening higher than 0.4% on each plate. IC₅₀ data were processed in a manner similar to that described for Example 2 to identify competitive, noncompetitive, or uncompetitive inhibitors of MMP-12CD.

### BINDING EXAMPLE 4

Fluorigenic Peptide-1 Substrate Based Assay for Identifying Competitive, Noncompetitive or Uncompetitive Inhibitors of MMP-2

In a manner similar to Example 2, an assay is run using about 1 Kd of acetohydroxamic acid (determined with MMP-2), wherein MMP-2 is substituted for MMP-13CD, to identify a competitive, noncompetitive, or uncompetitive inhibitor of MMP-2.

### BINDING EXAMPLE 5

Fluorigenic Peptide-1 Substrate Based Assay for Identifying Competitive, Noncompetitive, or Uncompetitive Inhibitors of MMP-9

In a manner similar to Example 3, an assay is run using about 1 Kd of acetohydroxamic acid (determined with MMP-9), wherein MMP-9 is substituted for MMP-12, with 0.22 nM enzyme and 20 μM compounds, to identify a competitive, noncompetitive, or uncompetitive inhibitor of MMP-9.

MMP-9 may be obtained from commercial sources. For example, full-length pro-MMP-9 purified from stimulated neutrophils is available from Calbiochem. Just prior to use, the enzyme should be activated using, for example, the activation protocol described in Example 1 for 0.01 μg/μl MMP-9 with 2 mM APMA incubated for 2 hours at 37° C.

### BINDING EXAMPLE 6

Fluorigenic Peptide-1 Substrate Based Assay for Identifying Competitive, Noncompetitive, or Uncompetitive Inhibitors of MMP-3

In a manner similar to Example 3, an assay is run using about 1 Kd of acetohydroxamic acid (determined with MMP-3), i.e. with 9 mM AcNHOH, wherein MMP-3 is substituted for MMP-12, with 2.2 nM enzyme and 20 μM compounds in an assay buffer comprising 50 mM Tri-Hcl pH 7.5 and 10 mM CaCl₂ to identify a competitive, noncompetitive, or uncompetitive inhibitor of MMP-3.

MMP-3 may be obtained from commercial sources. For example, full-length pro-MMP-3 is available from Calbiochem. Just prior to use, the enzyme should be activated using, for example, the activation protocol described in Example 1 for 1 μg/μl MMP-3 with 1 mM APMA incubated for 4 hours at 37° C.

Results from Examples 2, 4, and 5 for N-[(3-phenylisoxazol-5-ylmethyl)-aminothiocarbonyl]-benzamide are shown below in Table 2 in the column labeled “IC₅₀ Ratio (±).” In Table 2, noncompetitive or uncompetitive inhibitors that have an IC₅₀ Ratio (±) of less than 1 are synergistic with AcNHOH, which competitive inhibitors have an IC₅₀ Ratio (±) of greater than 1, unless otherwise indicated.

TABLE 2
Synergistic Inhibition of MMP-2, MMP-9, and MMP-13CD

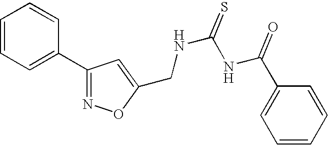

| | Without AcNHOH | With AcNHOH | |
|---|---|---|---|
| MMP | IC₅₀ (μM) | IC₅₀ (μM) | IC₅₀ Ratio (±) |
| 2FL | 0.9 | 0.09 | 0.10 |
| 9FL | 3.1 | 1.2 | 0.39 |
| 13CD | 2.3 | 0.2 | 0.09 |

Fluorigenic peptide (FP1) substrate assay
CD = catalytic domain
FL '2 full length protein

Steady-state kinetics of the mechanism of inhibition of MMP-2 by the compounds of Table 2 are shown in FIG. 8, and show the compound is an uncompetitive inhibitor of MMP-2. In FIG. 8, 1 over the initial velocity Vₒ, which is the initial rate of product formation in a steady state kinetics assay, is plotted on the Y-axis versus 1 over concentration of substrate [S] used in the assay on the X-axis for inhibition of MMP-2 with N-[(3-phenylisoxazol-5-ylmethyl)-aminothiocarbonyl]-benzamide at 6 concentrations of from 0 nanomolar to 600 nanomolar.

In principle, the method of Examples 1 to 6 may be readily adapted to identify competitive, noncompetitive, or uncompetitive inhibitors of any MMP, or a catalytic domain thereof, by substituting for MMP-13CD and the substrate of Example 1 or 2, the particular MMP, or a catalytic domain thereof, being screened against and an art-recognized substrate of the particular MMP, respectively. For example, the method has been adapted to identify competitive, noncompetitive, or uncompetitive inhibitors of any MMP-12CD. The invention method works using any mammalian MMP.

### BINDING EXAMPLE 7

Assays to Determine MMP Selectivity

The ability of a given compound to inhibit the activity of a given human MMP to hydrolyse fluorogenic substrates is determined using the following substrates and incubation conditions as referred to in the following publications.

| | | | Reaction | |
|---|---|---|---|---|
| MMP | Substrate | Incubation | product | Reference |
| Hmmp-1 | DMP-Pro-Cha-Gly- | 40 | Cys(Me)-His- | Bickett et al. |
| | Lys(Me)-His-Ala- | min./37° C. | Ala-Lys(n- | (1993) Anal. |
| | Lys(n-Me-Abz)-NH₂ | | Me-Abz)-NH₂ | Biochem. |
| | (10 μM) | | | 212: 58-64 |
| HMMP-2 | NFF-2 (6 μM) | 90 | Mca-Arg-Pro- | Nagase et al. |
| | | min./37° C. | Lys-Pro-Tyr- | (1994) J. Biol. |
| | | | Ala | Chem. |
| | | | | 269: 20952-7 |
| HMMP-3 | NFF-2 (10 μM) | 90 | Mca-Arg-Pro- | Nagase et al. |
| | | min./37° C. | Lys-Pro-Tyr- | (1994) J. Biol. |
| | | | Ala | Chem. |
| | | | | 269: 20952-7 |
| HMMP-7 | MMP-2/MMP-7 | 45 | Mca-Pro-Leu- | Quesada et al. |
| | substrate | min./37° C. | Gly | (1997) Clin. Exp. |
| | | | | Metastasis |
| | | | | 15: 339-40 |
| HMMP-8 | Mca-Pro-Cha-Gly- | 120 | Mca-Pro-Cha- | Knauper et al. |
| | Nva-His-Ala-Dpa- | min./22° C. | Gly | (1996) J. Biol. |
| | NH₂ (1 μM) | | | Chem. 271: |
| | | | | 1544-50 |
| HMMP-9 | NFF-2 (2 μM) | 90 | Mca-Arg-Pro- | Nagase et al. |
| | | min./37° C. | Lys-Pro-Tyr- | (1994) J. Biol. |
| | | | Ala | Chem. |
| | | | | 269: 20952-7 |
| HMMP-12 | FP1 substrate | 20 | Mca-Pro-Leu- | See Example 3 |
| | | min./20° C. | Gly-Leu | |
| HMMP-14 | MMP-14 substrate (2 | 60 | Mca-Pro-Leu- | Mucha et al. |
| | μM) | min./22° C. | Ala | (1998) J. Biol. |
| | | | | Chem. 273:2763- |
| | | | | 8 |

### Crystallization Conditions

Crystallization Conditions for Complexes of MMP13 CD (Catalytic Domain) and Allosteric Inhibitors—FIG. 7:

Recombinant human MMP13 catalytic domain (CD) was used at 5-12 mg/ml in 50 mM Tris, pH 7.6, 0.02 nM Zn₂Cl, 10 mM Ca₂Cl. Ternary complexes of MMP13 CD with inhibitors and acetohydroxamic acid were obtained by co-crystallization. The inhibitors were dissolved in DMSO (the final concentration of DMSO after mixing with protein did not exceed 5%) and mixed with diluted protein solution in at least 5:1 molar ration. 100 mM of acetohydroxamic acid solution was added to the complex followed by a hour incubation at 4° C. and centrifugated for 5 minutes at 3000 g. The supernatant was concentrated to 7-20 mg/ml. Crystallization was done in 2-4 μl hanging drops (1:1 ration of complex to reservoir solutions) equilibrated against 0.5 ml of the reservoir solution. Complexes with inhibitors were crystallized using 18-22% PEG MME 5K, 0.2 M Li₂SO₄, 0.1 M HEPES pH 7.0 as reservoir solution.

Crystallization Conditions for Complexes of MMP12 CD (Catalytic Domain) and Inhibitors of MMP12—FIG. 10/11:

Crystallization with Compound of Example 6—FIG. 10:

The complex is obtained as the same manner than the complex with MMP13 using MMP12 CD and compound of example 6. Complex with inhibitor is crystallized using 2.2-2.8 sodium chloride and 0.1 M imidazol pH 8.0, with a protein concentration at 9 mg/ml.

Crystallization with Compound of Example 7—FIG. 11:

The complex is obtained as the same manner than the complex with MMP13 using MMP12 CD and compound of example 7. Complex with inhibitor is crystallized using 0.1 M sodium Cacodylate pH 6.5 and 1.0-1.4 M sodium acetate, with a protein concentration at 10 mg/ml.

Crystallization Conditions for Complexes of MMP13 CD (Catalytic Domain) and Inhibitors of MMP12—FIG. 12:

The complex is obtained as the same manner than the complex with MMP13 using MMP13 CD and compound of example 8. Complex with inhibitor is crystallized using 10% (w/w) of PEG 8K and 9% (w/w) of PEG 1K.

These crystal structures show clearly that the compounds does not chelated the Zn atom and that they binds allosterically the MMPs through the S1′, or S1″, or S1′ and S1″ pocket(s).

### Appendix 2

### Pulmonary Disease Models

### REFERENCE EXAMPLE 12

Smoking Mice Model

Male A/J mice aged from 6 weeks known to present pulmonary susceptibility are challenged with tobacco smoke in order to induce emphysema. Animals are challenged with the smoke of 3 non-filtered cigarette (1R3; University of Kentucky, Lexington, Ky.) per chamber (0.25 m³, being able to contain 40 animals), twice a day, 30 minutes each time and 5 days a week using a large-whole body chamber. Nonsmoking, age-matched littermates are used as controls. Animals are then sacrificed at T0 (start of experiment), and at different time points such as T1 (Month 2) and T4 (Month 5).

### REFERENCE EXAMPLE 13

LPS-Stimulated Mice Model

Male 6-week old C57b1/6 mice were exposed in a plexiglass container to 100 μg/ml lipopolysaccharide or LPS (in a 0.9% pyrogen-free physiological sodium chloride solution) aerosol for 1 hour, delivered using the SPAG-2 series 6000 nebuliser system (ICN) with a flow rate of 8 liters per minute at a pressure of 26±2 psi. Non-exposed, age-matched littermates were used as controls. Animals (control and stimulated groups) were then sacrificed 72 h after the end of nebulisation.

1. A compound that is a matrix metalloproteinase inhibitor, and that:
(a) binds allosterically to said matrix metalloproteinase;
(b) binds into at least the S1′ pocket, at least the S1″ pocket or at least the S1′ pocket and the S1″ pocket of said matrix metalloproteinase; and
(c) exhibits selectivity for a matrix metalloproteinase or group of matrix metalloproteinases other than MMP-13.

2. A compound according to claim 1 that exhibits selectivity for MMP-1.

3. A compound according to claim 1 that exhibits selectivity for MMP-2.

4. A compound according to claim 1 that exhibits selectivity for MMP-3.

5. A compound according to claim 1 that exhibits selectivity for MMP-4.

6. A compound according to claim 1 that exhibits selectivity for MMP-7.

7. A compound according to claim 1 that exhibits selectivity for MMP-8.

8. A compound according to claim 1 that exhibits selectivity for MMP-9.

9. A compound according to claim 1 that exhibits selectivity for MMP-10.

10. A compound according to claim 1 that exhibits selectivity for MMP-11.

11. A compound according to claim 1 that exhibits selectivity for MMP-12.

12. A compound according to claim 1 that exhibits selectivity for MMP-14.

13. A compound according to claim 1 that exhibits selectivity for MMP-15.

14. A compound according to claim 1 that exhibits selectivity for MMP-16.

15. A compound according to claim 1 that exhibits selectivity for MMP-17.

16. A compound according to claim 1 that exhibits selectivity for MMP-19.

17. A compound according to claim 1 that exhibits selectivity for MMP-23.

18. A compound according to claim 1 that exhibits selectivity for MMP-24.

19. A compound according to claim 1 that exhibits selectivity for MMP-25.

20. A compound according to claim 1 that exhibits selectivity for MMP-26.

21. A compound according to claim 1 that exhibits selectivity for MMP-27.

22. A compound according to claim 1 that exhibits selectivity for MMP-28.

23. A compound according to claim 1 that exhibits selectivity for MMP-2 and MMP-9.

24. A compound according to claim 1 that exhibits selectivity for MMP-3 and MMP-9 and/or MMP-12.

25. A compound according to claim 1 that has an IC₅₀ for binding to its target matrix metalloproteinase or group of matrix metalloproteinases of less than 1 μM.

26. A compound according to claim 1 that has a selectivity for a single MMP target, or for a small number of MMP targets of at least 10 fold.

27. A compound according to claim 1 that has a selectivity for a single MMP target, or for a small number of MMP targets of at least 50 fold.

28. A compound according to claim 1 that has a selectivity for a single MMP target, or for a small number of MMP targets of at least 100 fold.

29. A compound according to claim 1 that has an IC₅₀ for binding to its target matrix metalloproteinase or group of matrix metalloproteinases that is not increased in the presence of a small molecule ligand for chelation to zinc.

30. The compound of claim 29 that has an IC₅₀ for binding to its target matrix metalloproteinase or group of matrix metalloproteinases that is not increased in the presence of acetohydroxamic acid.

31. The compound of claim 1 that has a molecular weight in the range 400-550 and comprises a monocyclic, bicyclc or tricyclic scaffold having 2-4 ring hetero atoms selected from N and S and carbonyl in at least one ring position, the scaffold being linked by first and second linking groups having 14 chain atoms to first and second monocyclic aryl or heteroaryl groups one or both of which have 1-2 polar or ionizable substituents or a heterocyclyl substituent.

32. The compound of claim 31, wherein the first linking group has 3 chain atoms and the second linking group has one chain atom.

33. The compound of claim 31, wherein the first linking group incorporates olefinic unsaturation, acetylenic unsaturation, carbonyl, ester or amide.

34. The compound of claim 31, wherein the polar or ionizable substituents are selected from alkoxy and carboxylate.

35. A pharmaceutical composition comprising a compound as claimed in claim 1 and a pharmaceutically acceptable carrier or diluent.

36. A method of treating or preventing a disease associated with over-expression of one or more matrix metaloproteinases in a patient suffering from, or liable to suffer from, said disease, which comprises administering to said patient a compound as claimed in claim 1.

37. The method of claim 36, wherein the disease results from breakdown of connective tissue and may be pulmonary disease, rheumatoid arthritis, osteoarthritis, osteoporosis, periodontitis, multiple sclerosis, gingivitis, corneal epidermal or gastric ulceration, atherosclerosis, neointimal proliferation, or tumor metastasis.

38. The method of claim 36, wherein the matrix metal proteinases are MMP-2 and MMP-9 and the patient is suffering from cancer.

39. The method of claim 36, wherein the matrix metal proteinases are MMP-3 and MMP-9 and the patient is suffering from, or liable to suffer from, rheumatoid arthritis or osteoarthritis.

40. The method of claim 36, wherein the matrix metal proteinase is MMP-9 or MMP-12 and the patient is suffering from, or liable to suffer from, chronic obstructive pulmonary disease or allergic rhinitis.

41. A method of identifying a compound as defined in claim 1, said method comprising:
docking the compound into the catalytic domain or domains of a target matrix metalloproteinase enzyme or group of such enzymes; and
checking the availability of a binding mode in which said compound binds allosterically into an S1′ pocket, S1″ pocket or both.

42. A method of identifying a compound as defined claim 1, said method comprising:
determining an IC₅₀ said compound for a target matrix metalloproteinase enzyme or group of such enzymes or group thereof other than MMP-13;
determining whether said compound exhibits selectivity for said target matrix metalloproteinase or group;
determining whether said compound binds allosterically.

43. A method of identifying a compound as defined in claim 1, said method comprising:
docking the compound into the catalytic domain or domains of a target matrix metalloproteinase enzyme or group of such enzymes;
checking the availability of a binding mode in which said compound binds allosterically into an S1′ pocket, S1″ pocket or both;
determining an IC₅₀ said compound for a target matrix metalloproteinase inhibitor or group thereof other than MMP-13;
determining whether said compound exhibits selectivity for said target matrix metalloproteinase enzyme or group of such enzymes;
determining whether said compound binds allosterically.

44. A method for identifying a matrix metalloproteinase inhibitor whose potency is relatively insensitive to concentration of substrate, said method comprising comparing the IC₅₀ of a test compound using a full-length MMP or a catalytic domain thereof in the presence and in the absence of a ligand that chelates readily to zinc but is a sufficiently small molecule that it does not substantially interfere with the binding of the inhibitor to its intended location adjacent to the catalytic domain.

45. The method of claim 43, wherein the ligand is acetohydroxamic acid.

46. The method of claim 43, wherein the ligand is acetic acid, propanoic acid, N-hydroxy-propanamide, acetoacetic acid, malonic acid, ethanethiol, 1,3-propanedithiol, N-hydroxy-benzamide, imidazole, 2-mercaptoethanol, cyanide, thiocyanate, 2,4,6-trihydroxypyrimidine, or 1,10-phenanthroline.

47. The method of claim 43, wherein the matrix metalloproteinase inhibitor is MMP12.


SEQUENCE LISTING (23 sequences)

SEQ ID NO: 1 - protein, 109 aa, Homo sapiens
  MMP 1
SFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPI

SEQ ID NO: 2 - protein, 111 aa, Homo sapiens
  MMP 8
AFYQRDHGDNSPFDGPNGILAHAFQPGQGIGGDAHFDAEETWTNTSANYNLFLVAAHEFGHSLGLAHSSDPGALMYPNYAFRETSNYSLPQDDIDGIQAIYGLSSNPIQPT

SEQ ID NO: 3 - protein, 119 aa, Homo sapiens
  MMP 3
SFAVREHGDFYPFDGPGNVLAHAYAPGPGINGDAHFDDDEQWTKDTTGTNLFLVAAHEIGHSLGLFHSANTEALMYPLYHSLTDLTRFRLSQDDINGIQSLYGPPPDSPETPLVPTEPV

SEQ ID NO: 4 - protein, 119 aa, Homo sapiens
  MMP 10
SFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSV

SEQ ID NO: 5 - protein, 111 aa, Homo sapiens
  MMP 13
SFGIKEHGDFYPFDGPSGLLAHAFPPGPNYGGDAHFDDDETWTSSSKGYNLFLVAAHEFGHSLGLDHSKDPGALMFPIYTYTGKSHFMLPDDDVQGIQSLYGPGDEDPNPK

SEQ ID NO: 6 - protein, 113 aa, Homo sapiens
  MMP 12
VFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYGDPKENQRLPNP

SEQ ID NO: 7 - protein, 118 aa, Homo sapiens
  MMP 11
DFARYWDGDDLPFDGPGGILAHAFFPKTHREGDVHFDYDETWTIGDDQGTDLLQVAAHEFGHVLGLQHTTAAKALMSAFYTFRYPLSLSPDDCRGVQHLYGQPWPTVTSRTPALGPQA

SEQ ID NO: 8 - protein, 123 aa, Homo sapiens
  MMP 14
FFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSR

SEQ ID NO: 9 - protein, 126 aa, Homo sapiens
  MMP 15
LFASGFHGDSSPFDGTGGFLAHAYFPGPGLGGDTHFDADEPWTFSSTDLHGNNLFLVAVHELGHALGLEHSSNPNAIMAPFYQWKDVDNFKLPEDDLRGIQQLYGTPDGQPQPTQPLPTVTPRRPG

SEQ ID NO: 10 - protein, 125 aa, Homo sapiens
  MMP 16
IFASGFHGDSSPFDGEGGFLAHAYFPGPGIGGDTHFDSDEPWTLGNPNHDGNDLFLVAVHELGHALGLEHSNDPTAIMAPFYQYMETDNFKLPNDDLQGIQKIYGPPDKIPPPTRPLPTVPPHRS

SEQ ID NO: 11 - protein, 282 aa, Homo sapiens
  MMP 2
NFGRWEHGDGYPFDGKDGLLAHAFAPGTGVGGDSHFDDDELWTLGEGQVVRVKYGNADGEYCKFPFLFNGKEYNSCTDTGRSDGFLWCSTTYNFEKDGKYGFCPHEALFTMGGNAEGQPCKFPFRFQGTSYDSCTTEGRTDGYRWCGTTEDYDRDKKYGFCPETAMSTVGGNSEGAPCVFPFTFLGNKYESCTSAGRSDGKMWCATTANYDDDRKWGFCPDQGYSLFLVAAHEFGHAMGLEHSQDPGALMAPIYTYTKNFRLSQDDIKGIQELYGASPDIDL

SEQ ID NO: 12 - protein, 297 aa, Homo sapiens
  MMP 9
QFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPP

SEQ ID NO: 13 - protein, 111 aa, Homo sapiens
  MMP 7
GFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 14 - protein, 300 aa, Artificial sequence
  Consensus
XFAXXXHGDXXPFDGPGGXLAHAXXPGPGIGGDAHFDXXEXWTXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXGXNLFLVAAHEXGHSLGXXHSXDPXALMXPXYXXXXXXXXFXLXXDDXXGIQXLYGXXXXXPXXXXXXXXXXPXXXX

SEQ ID NO: 15 - protein, 155 aa, Homo sapiens
  MMP 12
WRKHYITYRINNYTPDMNREDVDYAIRKAFQVWSNVTPLKFSKINTGMADILVVFARGAHGDFHAFDGKGGILAHAFGPGSGIGGDAHFDEDEFWTTHSGGTNLFLTAVHEIGHSLGLGHSSDPKAVMFPTYKYVDINTFRLSADDIRGIQSLYG

SEQ ID NO: 16 - protein, 155 aa, Homo sapiens
  MMP 13
WSKMNLTYRIVNYTPDMTHSEVEKAFKKAFKVWSDVTPLNFTRLHDGIADIMISFGIKEHGDFYPFDGPSGLLAHAFPPGPNYGGDAHFDDDETWTSSSAGYNLFLVAAHEFGHSLGLDHSKDPGALMFPIYTYTGKAHFMLPDDDVQGIQSLYG

SEQ ID NO: 17 - protein, 10 aa, Artificial sequence
  Common sequence motif
HXGHXXGXXH

SEQ ID NO: 18 - protein, 7 aa, Artificial sequence
  Fluorigenic peptide-1 substrate
PLGLXAR

SEQ ID NO: 19 - protein, 8 aa, Artificial sequence
  Substrate
XPXGKHAK

SEQ ID NO: 20 - protein, 4 aa, Artificial sequence
  Reaction product
CHAK

SEQ ID NO: 21 - protein, 6 aa, Artificial sequence
  Reaction product
RPKPYA

SEQ ID NO: 22 - protein, 7 aa, Artificial sequence
  Substrate
PXGXHAX

SEQ ID NO: 23 - protein, 4 aa, Artificial sequence
  Reaction product
PLGL